(19) United States
(12) Patent Application Publication
(10) Pub. No.: US 2023/0404463 A1
(43) Pub. Date: Dec. 21, 2023

(54) STIMULATION SYSTEM

(71) Applicant: Alphatec Spine, Inc., Carlsbad, CA (US)

(72) Inventors: Richard Arthur O'Brien, Cockeysville, MD (US); Robert Gerard Snow, Phoenix, MD (US); Kathryn Mae Turk, Carlsbad, CA (US)

(21) Appl. No.: 18/319,359

(22) Filed: May 17, 2023

Related U.S. Application Data
(60) Provisional application No. 63/343,941, filed on May 19, 2022.

(51) Int. Cl. A61B 5/388

(52) U.S. Cl. CPC A61B 5/388

(57) ABSTRACT

A method for facilitating acquisition of a motor evoked potential (MEP) may include facilitating an MEP stimulation sequence to obtain the MEP. The facilitating may include delivering a first stimulation pulse to one or more peripheral nerves of a patient. The method may also include delivering, after the facilitating, the MEP stimulation sequence to one or more cranial nerves of the patient to obtain the MEP. The method may also include determining, based on the MEP, a physiological response has occurred. The method may also include indicating that the physiological response has occurred. The facilitating may reduce an intensity of the train of stimulation pulses of the MEP stimulation sequence, limit movement of the patient to the region of interest during the delivery of the MEP stimulation, and improve an accuracy of determining that the physiological response has occurred.

### CROSS-REFERENCE TO RELATED APPLICATIONS

The present application claims priority to U.S. Provisional Application No. 63/343,941, filed May 19, 2022, and entitled, “Stimulation System,” the entirety of which is incorporated by reference herein.

### TECHNICAL FIELD

The subject matter described herein relates generally to patient monitoring and clinical neurophysiology, and more specifically to a stimulation system for detecting and identifying patient physiological responses using automated facilitated motor evoked potentials.

### BACKGROUND

Monitoring patients by recording waveforms in response to stimulation delivered to the patients during surgery allows for early identification and prevention of impending injuries, such as a nerve or spinal cord injury. In particular, motor-evoked potentials (“MEPs”) may be recorded during intraoperative monitoring for spinal and other types of surgery, to ensure integrity of the descending motor tracts. For example, stimulation may be cranially applied to a patient during spinal surgery to help ensure that no damage is caused to the patient's descending motor pathways and that no damage is caused to the motor functioning of the patient's limbs. Unfortunately, such responses may be affected by external factors such as level of anesthesia or use of paralytic agents requiring expert subjective interpretation. Generally, highly trained technologists under physician supervision may monitor patients to determine when MEPs should be obtained during an ongoing surgery and using sophisticated, multichannel amplifiers and display equipment, based on the results of MEPs, determine whether the stimulation resulted in a sufficient physiological response. Unfortunately, such expert personnel and equipment are costly and may be limited in their availability and/or require pre-booking. Such personnel may also need to manually optimize and modify stimulation parameters and subjectively analyze the waveforms during stressful circumstances, decreasing the accuracy, speed, objectivity, and efficiency in detecting physiological responses or changes in physiological responses, and thus leading to an increase in the risk of injury caused to patients during surgery.

Cranial stimulation to obtain MEPs can also disrupt the surgery and even injure the patient. For example, cranial stimulation to obtain MEPs generally causes all of the patient's limbs, body, jaw, and/or the like to have significant unwanted movement. Such movement can disrupt surgery, as the surgeon would need to stop performing the surgery while the stimulation is applied, prolonging the surgery. This may result in a reduced frequency of and thus reduced efficacy of testing. Such movement can also cause the patient's jaw to shut forcefully, causing oral damage to the patient. To obtain the desired physiological responses, cranial stimulation often has a very high voltage, which can also be detrimental to the patient. Moreover, obtaining MEPs can have a high rate of false positive alerting or false negative alerting. In other words, MEPs may often result in inaccurate detection of changes in or reasons for changes in physiological responses to the cranial stimulation. Accordingly, although obtaining MEPs during spinal surgery may be desirable, it often disrupts the surgery which can lead to under-utilization. Additionally, effects of anesthesia or paralytic agents on obtaining consistent waveforms and subjective interpretation and manual implementation may result in inaccuracies.

### SUMMARY

Systems, methods, and articles of manufacture, including computer program products, are provided for using automated or semi-automated facilitated MEPs to detect a physiological response.

According to some aspects, a method includes facilitating an MEP stimulation sequence to obtain an MEP. The facilitating includes delivering, via a first stimulation electrode, a first stimulation pulse to one or more peripheral or cranial nerves of a patient. The one or more peripheral or cranial nerves may be located within a region of interest. The first stimulation electrode may be coupled to the patient in the region of interest. The method may include delivering, after the facilitating and via a second stimulation electrode, the MEP stimulation sequence to one or more areas of the scalp or cranium of the patient to obtain the MEP response. The MEP stimulation sequence includes one or two trains of stimulation pulses. The second stimulation electrode is coupled to a scalp of the patient. The method may also include determining, based on the MEP result, whether a physiological response has occurred. The method may also include indicating, via a display coupled to the first stimulation electrode and the second stimulation electrode, that the physiological response has occurred. The facilitating is configured to one or more of: to optimize an intensity or number of pulses of the train or trains of stimulation pulses of the MEP stimulation sequence, limit movement of the patient to the region of interest during the delivery of the MEP stimulation and improve the accuracy of determining whether the physiological response has occurred.

In some aspects, the first stimulation pulse includes a single stimulation pulse.

In some aspects, delivering the first stimulation pulse to the one or more peripheral nerves of the patient is configured to be delivered by an independent stimulator or one used for somatosensory evoked potential (SSEP) stimulation.

In some aspects, the method includes delivering, prior to the facilitating and via the second stimulation electrode, a preceding MEP stimulation sequence to the cranium of the patient. The second MEP stimulation sequence includes a second train of stimulation pulses.

In some aspects, the preceding MEP stimulation sequence is delivered prior to the MEP stimulation sequence by an inter-train time interval. The facilitating occurs during the intertrain time interval.

In some aspects, the method includes delivering the MEP sequence within a period of time after the facilitation of the MEP stimulation sequence.

In some aspects, the period of time is predetermined.

In some aspects, the period of time is dynamically adjusted based on one or more parameters of the patient and/or a type of surgery being performed on the patient.

In some aspects, the facilitating occurs automatically within the period of time prior to the delivering the MEP stimulation sequence.

In some aspects, the facilitating is manually initiated prior to delivering the MEP stimulation sequence.

In some aspects, the facilitating is automatically initiated by the system in response to one or more inputs from an SSEP monitoring system, an EMG monitoring system, a heart rate monitoring system, a blood pressure monitoring system, an anesthesia level monitoring system, and/or other patient monitoring system.

In some aspects, one or more of the SSEP monitoring system, the EMG monitoring system, the heart rate monitoring system, the blood pressure monitoring system, an anesthesia level monitoring system and the other patient monitoring system is contained within or is a part of the stimulation system herein claimed.

In some aspects, the MEP includes one or more waveforms.

In some aspects, a processor stores the MEP as a baseline waveform.

In some aspects, the determining includes comparing the one or more waveforms of the MEP to the baseline waveform.

In some aspects, the determining whether the physiological response has occurred includes: comparing one or more features of the MEP to a threshold and/or detecting a presence of the physiological response when the one or more features is greater than or equal to the threshold.

In some aspects, the determining whether the physiological response has occurred includes: comparing one or more features of the MEP to a threshold and/or detecting a presence of the physiological response when the one or more features is less than or equal to the threshold.

In some aspects, the method includes an SSEP stimulation/acquisition system configured to facilitate the MEP stimulation sequence. The SSEP stimulation/acquisition system may be configured to acquire one or more SSEPs during or prior to delivery of the first MEP stimulation pulse. An MEP acquisition system may be configured to deliver the MEP stimulation sequence.

In some aspects, the MEP acquisition system includes the stimulation electrode and the recording electrode.

In some aspects, the second stimulation electrode includes four or more electrodes.

In some aspects, a method may be performed by an MEP detection system for detecting and identifying a patient physiological response. The method may include stimulating, using a first stimulation sequence and via a first stimulation electrode coupled to a patient, one or more areas of the scalp or cranium of the patient. The stimulating may be configured to generate an MEP. The method may include recording, via a first recording electrode coupled to the patient, a first plurality of resultant waveforms. The first plurality of resultant waveforms may represent a conditioned MEP.

The method may also include stimulating, using a second stimulation sequence and via a second stimulation electrode coupled to the patient, one or more peripheral or cranial nerves of the patient. The method may also include recording, via a first recording electrode coupled to the patient, a second plurality of resultant waveforms. The second plurality of resultant waveforms may represent a facilitated MEP.

The method may also include stimulating, using a third stimulation sequence and via the first stimulation electrode, the one or more areas of the scalp or cranium of the patient. The stimulating may be configured to generate an MEP. The method may also include recording, via the first recording electrode, a third plurality of resultant waveforms. The third plurality of resultant waveforms may represent an MEP.

The method may also include determining, based on the first, second, or third plurality of resultant waveforms, if a physiological response has occurred. The method may also include indicating, via a display coupled to the first stimulation electrode and the second stimulation electrode, if the physiological response has occurred.

In some aspects, the first stimulation sequence includes a first plurality of stimulation pulses. The second stimulation sequence includes a single or two stimulation pulses. The third stimulation sequence includes a second plurality of stimulation pulses.

In some aspects, the first stimulation sequence is configured to condition or prime the one or more areas of the brain for obtaining the conditioned MEP.

In some aspects, the second stimulation sequence is configured for obtaining the facilitated MEP.

In some aspects, the third stimulation sequence is configured for obtaining the MEP.

In some aspects, the first stimulation electrode is coupled to a scalp of the patient. The second stimulation electrode is coupled to a peripheral or cranial nerve region of interest of the patient. The peripheral region of interest includes one or more of a limb, a hand, a wrist, a leg, and a foot of the patient. The cranial nerve region includes one or more areas of the face.

In some aspects, the first stimulation sequence is delivered prior to the third stimulation sequence separated by an inter-train time interval. The second stimulation sequence occurs during the inter-train time interval for obtaining a conditioned and facilitated MEP.

In some aspects, the first simulation sequence is omitted but the second and third sequences are delivered for obtaining a facilitated MEP.

In some aspects, the second stimulation sequence may be delivered prior to the first and third stimulation sequences.

In some aspects, the second stimulation sequence may be delivered to more than one peripheral or cranial nerve region of interest of the patient either simultaneously or in rapid succession. The peripheral region of interest includes one or more of a limb, a hand, a wrist, a leg, and a foot of the patient. The cranial nerve region includes one or more areas of the face.

In some aspects, the method includes stimulating using the third stimulation sequence within a period of time after the stimulating using the second stimulation sequence for obtaining the facilitated MEP.

In some aspects, the period of time is predetermined.

In some aspects, the period of time is dynamically adjusted based on one or more parameters of the patient and/or a type of surgery being performed on the patient.

In some aspects, the stimulating using the second stimulation sequence occurs automatically at a predetermined time between the stimulating using the first stimulation sequence and the stimulating using the third stimulation sequence for obtaining the facilitated MEP.

In some aspects, the determining includes comparing the plurality of waveforms to a baseline waveform.

In some aspects, determining if the physiological response has occurred includes comparing one or more features of the MEP to a threshold and detecting a presence of the physiological response when the one or more features is greater than or equal to the threshold. The threshold may include but is not limited to amplitude, morphology, latency, signal to noise ratio or an automated waveform detection classifying algorithm.

In some aspects, determining if the physiological response has occurred includes comparing one or more features of the MEP to a threshold and detecting a presence of the physiological response when the one or more features is less than or equal to the threshold. The threshold may include but is not limited to amplitude, morphology, latency signal to noise ratio or an automated waveform detection classifying algorithm.

In some aspects, the second stimulation electrode coupled to the patient may include utilizing one or more stimulating electrodes also used for SSEPs as the first stimulating electrode to deliver the second stimulation sequence.

According to some aspects, a method for detecting and identifying a first physiological response may include acquiring one or more first SSEPs. The acquiring may include stimulating, via a first stimulation electrode, one or more peripheral or cranial nerves of the patient at a region of interest. Then determining, by various means, if the somatosensory evoked potential may have changed from previous acquisitions. Then automatically acquiring a motor evoked potential (MEP) may include stimulating, via a second stimulation electrode, to one or more areas of the scalp or cranium of the patient. The method may include determining, based on the MEP, whether a change in the SSEP physiological response is accurate. The MEP response indicates one or both of whether the patient has motor function in the region of interest and to what extent. The method includes indicating, via a display coupled to the first stimulation electrode and the second stimulation electrode, that the physiological response has occurred.

The method includes acquiring one or more second SSEPs. The acquiring step includes stimulating, via the first stimulation electrode, the one or more peripheral nerves of the patient. The one or more second SSEPs indicates continuity of the sensory pathway.

Implementations of the current subject matter can include methods consistent with the descriptions provided herein as well as articles that comprise a tangibly embodied machine-readable medium operable to cause one or more machines (e.g., computers, etc.) to result in operations implementing—or signaling the need to implement—one or more of the described features. Similarly, computer systems are also described that may include one or more processors and one or more memories coupled to the one or more processors. A memory, which can include a non-transitory computer-readable or machine-readable storage medium, may include, encode, store, or the like one or more programs that cause one or more processors to perform one or more of the operations described herein. Computer implemented methods consistent with one or more implementations of the current subject matter can be implemented by one or more data processors residing in a single computing system or multiple computing systems. Such multiple computing systems can be connected and can exchange data and/or commands or other instructions or the like via one or more connections, including, for example, to a connection over a network (e.g. the Internet, a wireless wide area network, a wireless peer-to-peer network, a local area network, a wide area network, a wired network, or the like), via a direct connection between one or more of the multiple computing systems, etc. The current implementation may take into account or further utilize other methods of producing optimized MEP responses including multi-polar stimulation (Quadripolar, Sextupolar, etc.).

The details of one or more variations of the subject matter described herein are set forth in the accompanying drawings and the description below. Other features and advantages of the subject matter described herein will be apparent from the description and drawings, and from the claims. The claims that follow this disclosure are intended to define the scope of the protected subject matter.

### DESCRIPTION OF DRAWINGS

The accompanying drawings, which are incorporated into and constitute a part of this specification, show certain aspects of the subject matter disclosed herein and, together with the description, help explain some of the principles associated with the disclosed implementations. In the drawings,

FIG. 1 depicts a system diagram illustrating a stimulation system, consistent with implementations of the current subject matter;

FIG. 2 depicts a functional block diagram of a system for monitoring nerve function, consistent with implementations of the current subject matter;

FIG. 3 depicts an example user interface, consistent with implementations of the current subject matter;

FIG. 4 depicts an example stimulation sequence, consistent with implementations of the current subject matter;

FIG. 5 depicts an example stimulation sequence, consistent with implementations of the current subject matter;

FIG. 6 depicts a flowchart illustrating a process for detecting and identifying a patient physiological response, consistent with implementations of the current subject matter;

FIG. 7 depicts a flowchart illustrating a process for detecting and identifying a patient physiological response, consistent with implementations of the current subject matter;

FIG. 8 depicts a flowchart illustrating a process for detecting and identifying a patient physiological response, consistent with implementations of the current subject matter; and

FIG. 9 depicts a block diagram illustrating a computing system, in accordance with some example implementations.

When practical, similar reference numbers denote similar structures, features, or elements.

### DETAILED DESCRIPTION

Monitoring patients by recording waveforms in response to stimulation delivered to the patients during surgery allows for early identification and prevention of impending injuries, such as a nerve injury or spinal cord injury. This may include stimulation proximally at the head or spinal cord (central nervous system) with measuring muscle responses distally (general motor pathway monitoring) or stimulating peripherally and measuring responses more proximally over the central nervous system (general sensory pathway monitoring)

Generally, the pyramidal nervous system is the motor system under test during intraoperative neuromonitoring. The pyramidal nervous system includes two primary sets of motor neurons: Upper Motor Neurons (UMN) and Lower Motor Neurons (LMN). The excitability of the UMNs and LMNs, or their likelihood to respond to stimulation, is modulated by input on the stimulation system from central sources (e.g., brain, spinal cord, cerebellum and/or the like) and peripheral sources (e.g., sensory sensors).

While somatosensory evoked potentials (SSEPs)—which may include the electrical signals generated by a patient's nervous system in response to an electrical stimulus applied to a peripheral or cranial nerve of the patient—allow direct and ongoing testing of the ascending sensory pathways (e.g., the posterior columns in the spinal cord) and are effective in certain surgeries, the major motor pathways in the spinal cord obtain their blood supply from a different source—the anterior spinal arteries. Thus, where injury is localized to only the anterior spinal artery distribution, SSEPs may remain intact while significant motor pathway dysfunction occurs. Motor evoked potentials (MEPs)—which include the electrical signals generated by a patient's nervous system in response to an electrical or magnetic stimulus typically applied to the cranium or brain—allows for direct testing of the somatic motor pathways, indicating whether the patient has the ability to move a region of interest, such as the patient's limbs (e.g., arms, legs) and/or extremities (e.g., hands, wrists, feet, and/or the like) or face. Thus, monitoring MEPs during surgery can help to prevent damage to the patient's nerves that control the motor functioning of the patient.

MEPs may be recorded during intraoperative monitoring for spinal surgery, to ensure integrity of the descending motor tracts. These may include one or more series of stimuli, each of which may contain one or more stimuli. The stimulation system described herein may provide for facilitated MEP stimulation and acquisition. For example, the stimulation system described herein may stimulate one or more peripheral or cranial nerves of a patient prior to or between series of stimulating one or more cranial positions on the patient to obtain the MEP. Such stimulation requires precise timing between the peripheral and following central stimulus or stimulus series. Inaccurate or inappropriate intervening time may be counter-productive and result in suppression rather than accentuation of the resultant MEP response. In addition, this timing varies somewhat between patients and recording sites.

The intervening time between the peripheral or cranial nerve stimulation and the cranial stimulation or stimulation series may be determined or optimized automatically by calculation using one or more of the patient's height, the SSEP latency from the same or adjacent peripheral stimulator, the latency from an un-facilitated MEP cranial stimulation, or by measuring motor suppression or facilitation from specific pre-chosen intervening times

The stimulation intensity required to obtain a facilitated MEP is typically less than that for a non-facilitated MEP and can be steered to provide a response in a particular side or limb of interest. It can be used in conjunction with other methods of reducing overall stimulation intensity such as additional series of cranial stimulation, multi-polar cranial stimulation or image guided cranial stimulation. Its use can also be tailored to the type, dose, and duration of anesthesia and paralytic being used.

Facilitating the MEP stimulation and acquisition by, for example, first stimulating one or more peripheral nerves, may reduce the need for a technologist in the operating room, as it may reduce disruptions during the surgical procedure, reduce patient movement during MEP acquisition, reduce a voltage of the stimulation applied to the patient during MEP acquisition, steer stimulation to only the anatomical area of risk or interest, allow more frequent testing to reduce time between change detection, reduce false positives and/or false negatives in physiological response detection, allow better trending of MEP responses, and/or the like.

MEPs actuate the pyramidal nervous system by stimulating the upper motor neurons and recording resultant electrical waveforms from muscles. Stimulation for MEPs falls into two categories: Electrical and Magnetic, referred to as ‘Trans-Cranial Simulation Electrical MEPs (TSE-MEP or sometimes TceMEP) or Trans-Cranial Stimulation Magnetic MEPs (TSM-MEP or sometimes TcmMEP). The stimulation causes the upper motor neurons to produce an internally generated descending volley of depolarization, which in turn activates the lower motor neurons through an inter-neuron synapse, thereby activating the patient's muscles through the neuromuscular junction (NMJ). Stimulation of the brain, such as via one or more cranial areas, may induce three types of responses: (1) D waves that represent direct activation of the motor cortex and direct recording from the spinal cord; (2) I waves that are recorded from the spinal cord and result from cortical interneuron reactivation; and (3) M waves that are recorded from muscle activation. Suppression of the volley may thus occur at the descending UMN tract, the UMN to LNM synapse or interneuron, the LMN tract or supplied nerve, the NMJ or the muscle itself, and/or the like.

Generally, stimulation electrodes are positioned over a portion or portions of the scalp or cranium of the patient, under which the motor cortex containing the representation of the regions of interest lies. The stimulation electrodes deliver stimulation to the cranium. The stimulation may be of constant voltage and/or constant current and may include a train of stimulation pulses including a plurality of stimulation pulses. MEP responses are recorded, because of the stimulation, as compound motor action potentials (CMAP) using electrical or mechanomyography sensors. MEPs are generally acquired prior to and/or after a risky surgical maneuver to ensure that the patient maintains motor functioning of the patient's regions of interest but are most useful if recorded more frequently, allowing more timely identification of changes.

The use of MEPs may present certain advantages over the use of SSEPs. For example, MEPs directly measure the corticospinal pathway, provide instant results without having to average waveforms, may be less susceptible to interference from peripheral neuropathies, and can be less sensitive to electrical noise than SSEPs. But although MEPs can be used in conjunction with or when SSEPs are not available, MEPs can be difficult to rely on due to their intermittent testing nature, sensitivity to certain anesthetic and paralytic agents and tendency to disrupt surgical procedures. For example, to record MEPs, high voltage stimulations are generally applied to the patient's cranium or other areas of the central nervous system. This can cause significant and unwanted patient movement, including a significant portion of the patient's body—such as the patient's limbs, extremities, neck, jaw, and/or the like—that are not areas of interest for monitoring. This presents risks to the patient. The patient may have unwanted neck flexion, such as during cervical surgeries. The patient's jaw may close forcefully causing oral damage. Such movement can significantly disrupt the surgery. Accordingly, if an MEP is desired, surgeons often pause the surgery due to the significant, unwanted patient movement that comes with collecting the MEP. Thus, surgeons may be reluctant to perform them frequently.

Moreover, MEPs can have other disadvantages. For example, trained technologists, such as trained technologists under physician supervision, may need to be present to monitor patients during surgery using sophisticated, multichannel amplifiers and display equipment. Such personnel and equipment are costly and may be limited in their availability or require pre-booking. The personnel may also be prone to subjectivity when analyzing the waveforms under stressful situations, decreasing the accuracy, speed, and efficiency in producing valid MEPs and in detecting changes in physiological responses, and thus leading to unwanted interruptions or even an increase in injuries caused to the patient during surgery. Such systems may also rely on the technologist to alert the surgeon to stop the surgery before stimulating the patient to obtain the MEP. Subjectively monitoring the signals in real-time may lead to false alerts, and/or inaccurately identifying a patient physiological response, which may lead to an increased risk of injuries caused to the patient during surgery. MEPs are also very sensitive to the effects of some anesthetic agents which may produce unexpected changes or changes that are difficult to differentiate from tissue dysfunction due to the surgery itself. Generally, MEPs can result in a large number of delayed or false positive detections of physiological responses.

MEPs also provide only a snapshot of the electrical waveforms, rather than continuous data. Thus, it may be difficult to obtain an accurate account of the state of the patient's nervous system between the acquisition of different MEPs.

The stimulation system described herein may provide for facilitated MEP stimulation. In other words, the stimulation system may deliver stimulation to one or more peripheral or cranial nerves of the patient prior to, during or between series of delivering stimulation to one or more cranial stimulators on the patient to obtain the MEP. Such facilitated MEP stimulation, as described herein, may desirably steer the provoked muscle response to the region of interest of the patient's body where the one or more peripheral nerves are stimulated. Such steering may be automatically applied according to the type or anatomical location of the surgery and the associated at-risk neural structures. Such facilitated MEP stimulation as described herein may additionally and/or alternatively reduce the overall stimulation intensity of the stimulation or number of stimuli delivered to the patient's scalp, brain, or cranium, as the stimulation is focused to the region of interest. The system described herein may automatically calculate the association between a non-facilitated and facilitated MEP to accurately determine the appropriate alerting criteria for a change in a facilitated MEP. The facilitated MEP stimulation described herein may additionally and/or alternatively allow for testing of MEPs more frequently and to be activated by the surgeon and/or automatically after a change in SSEPs or other modality to confirm or deny a significant new or impending injury, thereby providing the surgeon with more continuous information.

The facilitated MEP stimulation system described herein may additionally and/or alternatively result in fewer disruptions of a surgical procedure. For example, due to the reduced patient movement, surgical procedures may not need to be stopped to obtain the MEP, resulting in more frequent testing and quicker and safer surgical procedures. The facilitated MEP stimulation described herein may additionally and/or alternatively reduce false positives and/or false negatives in detecting physiological responses. For example, the automated facilitation of the MEP stimulation and delivery of the MEP stimulation may reduce or eliminate the need for technologists to manually initiate the MEP detection. Moreover, in the most difficult cases, such as when a false positive or false negative finding occurs and surgeons may need to resort to waking the patient to determine whether the patient has motor functioning in the region of interest, the facilitated MEP stimulation described herein may more accurately detect physiological responses, reducing or eliminating such cases.

As described herein, the stimulation system may identify one or more patient physiological responses (e.g., motor functioning or motor responses of the patient) from recorded waveforms and use information from other EPs to determine when MEP stimulation is necessary to clarify the significance of a change in one of those modalities or failure to obtain responses from one of those modalities. The patient physiological responses may include one or more evoked potentials (EPs), such as somatosensory evoked potentials (SSEPs), auditory evoked potentials (AERs), motor evoked potentials (MEPs), brain stem auditory evoked potentials (BAEPs), and/or visual evoked potentials (VERs), among others. EPs may include any potential recorded from the nervous system, resulting from the application of a stimulus to a portion of the patient's body. For example, an EP may include a voltage versus time signal obtained by ensemble averaging the electrophysiological responses to repetitive stimulation of a specific sensory neural system detected using suitable electrodes. In some instances, where the signal to noise ratio is better, the electrophysiological responses are not ensemble averaged.

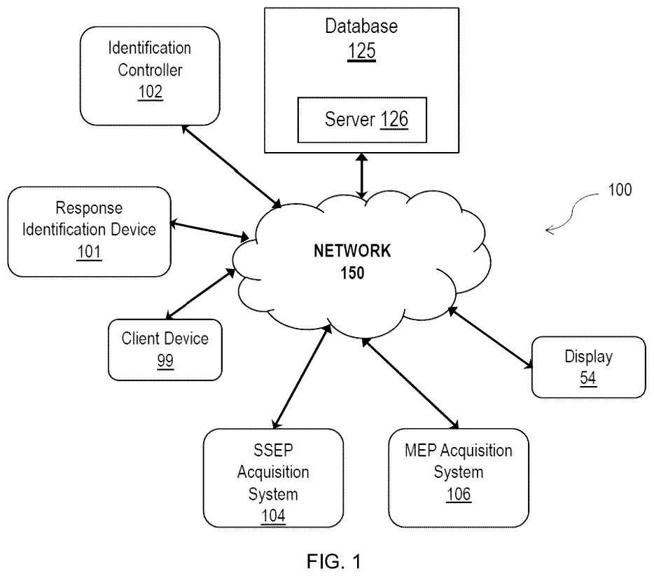

FIG. 1 depicts a system diagram illustrating a stimulation system 100, in accordance with some example implementations. Referring to FIG. 1, the stimulation system 100 may include a display 54, a client device 99, an identification controller 102, a database 125, a response identification device 101, an SSEP acquisition system 104, and/or an MEP acquisition system 106. In some example implementations, the display 54, the client device 99, the identification controller 102, the SSEP acquisition system 104, the MEP acquisition system 106, and/or the database 125 may form a portion of a response identification device 101 and/or may be positioned within a housing of the response identification device 101.

Referring to FIG. 1, the response identification device 101, the display 54, the client device 99, the identification controller 102, the SSEP acquisition system 104, the MEP acquisition system 106, and/or the database 125 may be communicatively coupled via a network 150 and/or via a direct device-device connection as described herein. The link or network 150 may be a wired and/or wireless network including, for example, a public land mobile network (PLMN), a local area network (LAN), a virtual local area network (VLAN), a wide area network (WAN), the Internet, a short-range radio connection, for example Bluetooth®, a peer-to-peer mesh network, and/or the like.

The client device 99 may be a mobile device such as, for example, a smartphone, a laptop computer, a tablet computer, a wearable apparatus, and/or the like. However, it should be appreciated that the client device 99 may be any processor-based device including, for example, a desktop computer, a laptop or mobile computer, a workstation, and/or the like. For example, via the client device 99, the clinician may be able to configure certain parameters of the response identification device 101, such as a stimulation sequence or stimulation strength, a response recording protocol, and the like. In some implementations, the client device 99 forms a part of the response identification device 101. Additionally, in some examples, via the client device 99, the user may configure various stimulation or protocols, and/or the like.

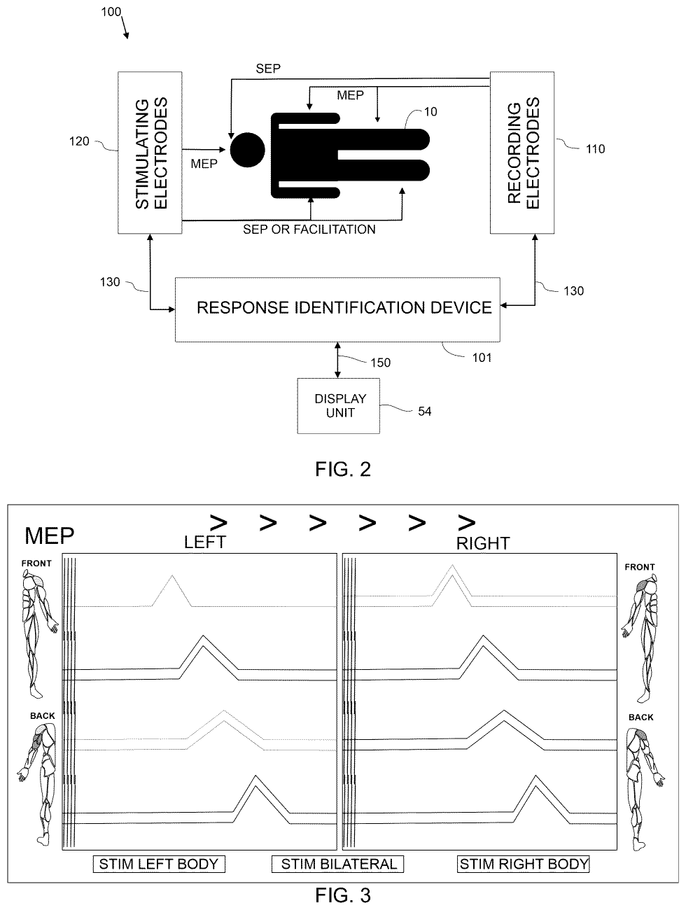

Referring to FIG. 2, the stimulation system 100 may include the response identification device 101, one or more recording electrodes 110 and/or one or more stimulation electrodes 120 coupled to a patient 10, and the display 54.

Referring to FIG. 2, the response identification device 101 of the stimulation system 100 may include integrated portions devoted to various modalities including but not limited to MEP and SSEP and a response identification controller to allow such integration.

The stimulation electrodes 120 may be positioned peripherally on or near the arms or legs of the patient 10 over peripheral nervous structures such as, the ulnar nerves, median nerves, peroneal nerves, saphenous nerves, and/or posterior tibial nerves and over the scalp or cranium to deliver a central stimulus.

The stimulation electrodes 120 may be intended for placement on a patient's skin on the wrists and/or ankles so that the electrodes are located over or near for example the ulnar nerves and posterior tibial nerves and over the scalp or cranium to deliver a central stimulus. These configurations, such as to obtain SSEPs, allow for full patient monitoring of peripheral nerves and sensory tracts, such as monitoring of the nerves in all limbs and posterior columns of the spinal cord of the patient 10. In some implementations, the stimulation system 100 may be used for upper limb monitoring. In such implementations, the stimulation electrodes 120 may be intended for placement on the skin of a patient's wrists, for example, over or near the ulnar or median nerves. These configurations allow for full patient monitoring of the sensory system, such as to obtain SSEPs and detect sensory functioning of the patient's limbs, extremities, arms, legs, feet, hands, wrists, neck, face and/or the like.

In some implementations, such as to obtain the MEP, the stimulation electrodes 120 may be positioned on the cranium of the patient over nervous structures, such as the motor cortex. The stimulation electrodes 120 may be intended for placement on a patient's skin on the scalp or cranium of the patient. These configurations allow for full patient monitoring of pyramidal motor system, such as to obtain MEPs and detect motor functioning of the patient's limbs, extremities, arms, legs, feet, hands, wrists, neck, face and/or the like.

The recording electrodes 110 may be positioned over the trunk, spine, neck, and/or head of the patient 10 to record signals from the peripheral nerves or brain. In some implementations, the recording electrodes 110 are intended to be placed on the skin on or over the truncal muscles or facial muscles, of the patient 10. In some implementations, the recording electrodes 110 may be positioned over muscles, particularly those muscles related to the nerve level near where a surgical procedure is to be performed.

In some embodiments, stimulating electrodes may be placed both at or near the cranium while only one set of recording electrodes is placed on or near the muscle(s) of interest.

As shown in FIG. 2, the response identification device 101 may be coupled to the recording electrodes 110 and the stimulation electrodes 120, such as via a plurality of cables 130. The response identification device 101 may also be electrically, electronically, and/or mechanically coupled to the display 54, such as via a link 150. The link 150 may include internal wiring and/or an external cable. In some implementations, the link 150 is a wireless communication link. For example, the response identification device 101 may be wirelessly coupled to the display 54 via Bluetooth® or other radiofrequency signal or via near field communications or a cellular signal.

The SSEP acquisition system 104 of the response identification device 101 may apply electrical stimulation to peripheral nerves of a patient by sending electrical signals to the stimulation electrodes 120 located on some or all of a patient's limbs or cranial nerves. Repeated stimulation elicits a response of the patient's nervous system in the form of physiological responses, such as EPs, which travel up the peripheral or cranial nerves, for instance, through the dorsal column of the spinal cord, and to the brain. EPs may be detected and changes in the monitored EP may indicate changes in nerve function. For example, the recording electrodes 110 may receive one or more resultant electrical waveforms in response to stimulation being provided to the patient 10 via the stimulation electrodes 120. The response identification device 101 (e.g., via The SSEP acquisition system 104) may detect changes, such as changes in latency, changes in amplitude, or changes in morphology, in the EPs. Based on the observed changes, the response identification device 101 (e.g., via The SSEP acquisition system 104) may identify potential injuries caused by a physical position of the patient's body, an effect of the procedure being performed, the stimulation being delivered to the patient, and/or the like. In some implementations, the response identification device 101 (e.g., via The SSEP acquisition system 104) identifies a particular nerve structure or body region affected by positioning effect or the stimulation based on the EPs. The response identification device 101 (e.g., via The SSEP acquisition system 104) may additionally and/or alternatively recommend actions, such as via the display 54, to ameliorate the injuries by recommending changes in position.

The MEP acquisition system 106 of the response identification device 101 may apply electrical stimulation to a patient by sending electrical signals to the stimulation electrodes 120 located on the patient's cranium or scalp. Repeated stimulation elicits a response of the patient's nervous system in the form of physiological responses, such as EPs, which travel through the nervous system to at least the region of interest or at risk of injury of the patient. EPs may be detected and changes in the monitored EP may indicate changes in nervous system function. For example, the recording electrodes 110 may receive one or more resultant electrical waveforms in response to stimulation being provided to the patient 10 via the stimulation electrodes 120. The response identification device 101 (e.g., via The MEP acquisition system 106) may detect changes, such as changes in latency, changes in amplitude, or changes in morphology, in the EPs. Based on the observed changes, the response identification device 101 (e.g., via The MEP acquisition system 106) may identify potential injuries to the patient's nervous system and/or potential reduced motor functioning of the patient's regions of interest. The response identification device 101 (e.g., via The MEP acquisition system 106) may additionally and/or alternatively recommend actions, such as via the display 54, to ameliorate or mitigate the injuries.

As noted above, the stimulation system 100 may include one or more stimulation electrodes 120. It may include one or more stimulating systems linked temporally. The response identification device 101 may sequentially stimulate peripheral nerves of the patient 10 via the stimulation electrodes 120 while recording the EPs via the recording electrodes 110. cranial stimulations, stimulating the peripheral nerves of the patient 10, the response identification device 101 may also stimulate the cranium of the patient 10 via the stimulation electrodes while recording the EPs via the recording electrodes 110. Thus, the stimulation of the peripheral nerves prior to the stimulation of the cranium may facilitate the stimulation of the cranium in obtaining the MEP. Such peripheral stimulation may also occur between two or more series of cranial stimulations. Accordingly, in some implementations, the stimulation electrodes 120 are coupled to the response identification device 101 as an output, and the recording electrodes 110 are coupled to the response identification device 101 as an input.

The response identification device 101 may include various circuitry components, such as electric stimulators, pre-amplifiers, amplifiers and/or other components, to control stimulation and process the return signals. In some implementations, the response identification device 101 may average together the response to several stimuli to reduce noise in the signal.

As described herein, the response identification device 101, such as via the response identification controller portion, may analyze signals and determine when warnings and alerts are appropriate. For example, the response identification device 101 may send signals to the display 54 to display warnings and/or alerts, such as when the stimulation is approaching a nerve of the patient, when a patient has motor functioning in regions of interest, and/or when the patient loses or has impaired motor functioning in the regions of interest.

The display 54 may form a part of the response identification device 101 and/or the client device 99, and/or may be separately coupled to the response identification device 101 and/or the client device 99. The display 54 may also include a user interface. The user interface may form a part of a display screen of the display 54 that presents information to the user (e.g., a clinician, a patient, a technologist, and/or the like) and/or the user interface may be separate from the display screen. For example, the user interface may include one or more buttons, or portions of the display screen that are to receive an entry from the user.

The display 54 may display various information, such as biographical information of a patient, suggested locations of electrodes, stimulation parameters, areas being stimulated and recorded, baseline and current signal traces, historical trends in signals, relevant changes in signals, location of signal changes, quality of recorded signals, position of electrodes, alerts due to significant changes in signals, proposed movements to mitigate detrimental signal changes, recorded resultant electrical waveforms, and/or the like. The display 54 may allow an operator to set up the initial monitoring layout or plan and interact with the display 54 during monitoring to add additional information, view information in a different format, and/or respond to alerts. In some implementations, the display 54 may allow override of a change in signal by a user, such as when a signal change is related to a change in dose of anesthetic agent or some other event unrelated to the stimulation of the nerves or cranium of the patient.

FIG. 3 illustrates an example of the display 54, consistent with implementations of the current subject matter. In some implementations, the stimulation system 100 facilitates setup of the stimulation protocol by a clinician and/or non-expert personnel by providing visual cues and instructions during the setup process. For example, as shown in FIG. 3, the display may show MEP responses either as physiological waveforms or interpreted pictograms of muscle responses. The the display 54 may also display pictorial instructions of where to place stimulating and/or recording electrodes, such as the stimulation electrodes 120 and/or the recording electrodes 110, on a patient's body. Such an image may appear at startup of the response identification device 101, upon indicating that monitoring of a new patient is commencing, or upon receiving a signal that a cable has been connected to the response identification device 101.

Generally, the display 54 (e.g., a dynamic display) also improves the manner in which the client device 99 and/or the response identification device 101 displays information and interacts with the user. By dynamically generating values based on an input, the client device 99, and/or the response identification device 101 may reduce the need to render additional complex data entry elements to complete programing. For example, the graphical user interface presented by the display 54 may include graphical elements to increment or decrement a value of the displayed parameters rather than presenting a full keypad for data entry. The client device 99 and/or the response identification device 101 may more efficiently process and validate these input signals, which may be more than entries from freeform text or numeric data entry fields. The use of smaller entry elements also conserves display area on the client device 99 and/or the response identification device 101. This permits presentation of more programming parameters at the time of data entry thereby further reducing the likelihood of a programming error.

Referring back to FIG. 1, the database 125 may include one or more databases, providing physical data storage within a dedicated facility and/or being locally stored on the response identification device 101 and/or the client device 99. Additionally, and/or alternatively, the database 125 may include cloud-based systems providing remote storage of data in, for example, a multi-tenant computing environment and/or the like. The database 125 may also include non-transitory computer readable media. The database 125 may store data recorded from and/or calculated based on the waveforms recorded by the recording electrodes 110 and/or received by the response identification device 101.

The database 125 may include and/or be coupled to a server 126, which may be a server coupled to a network, a cloud server, and/or the like. The response identification device 101 and/or the client device 99 may wirelessly communicate with the server 126. The server 126, which may include a cloud-based server, may provide and/or receive data and/or instructions from the data system 125 to the response identification device 101 and/or the client device 99, to implement one or more features of the stimulation system 100, consistent with implementations of the current subject matter. Additionally, and/or alternatively, the server 126 may receive data (e.g., one or more waveform signals, patient information, information characterizing the one or more waveform signals, and/or the like) from the response identification device 101 and/or the client device 99.

The identification controller portion may be at least partially embedded and/or implemented within the response identification device 101 and/or the client device 99. The controller 102 may detect and identify, based on the recorded waveforms, a patient physiological response to help prevent or reduce the risk of injury caused to the patient's nerves during surgery.

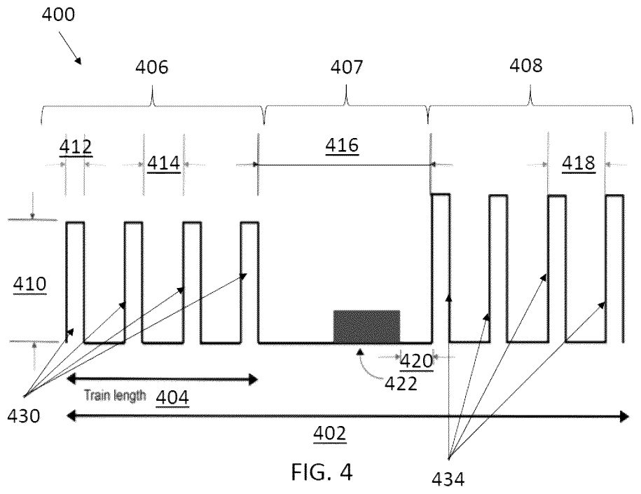

Consistent with implementations of the current subject matter, central stimulation to obtain an MEP may be conditioned or facilitated or both by delivering a prior stimulation cranially (cranial conditioning stimuli) or peripherally to one or more peripheral nerves of the patient (facilitation). FIG. 4 illustrates an example stimulation sequence 400, consistent with implementations of the current subject matter, that may be implemented automatically by system 100 or may be implemented by system 100 upon request by a user. The stimulation sequence 400 may include one or more (e.g., one, two, three, four, five, six, seven, eight, nine, ten, or more) stimulation sequences or trains. In other words, a number of stimulation trains 402 of the stimulation sequence 400 may be one, two, three, four, five, six, seven, eight, nine, ten, or more stimulation trains. As shown in FIG. 4, the stimulation sequence 400 may include a first stimulation sequence 406, a second stimulation sequence 407, and a third stimulation sequence 408. In some implementations, before, during, or after the stimulation sequence 400, the system 100, such as via the SSEP acquisition system 104, may acquire one or more SSEPs by stimulating one or more peripheral nerves of the patient. While the stimulation sequence 400 generally depicts acquisition of an MEP, such as via the MEP acquisition system 106, SSEPs may be acquired prior to the acquisition of the MEP using the sequence 400. Facilitation stimulations 422 may be delivered as a part of the second stimulation sequence 407 by use of the SSEP stimulating electrodes or separate stimulating electrodes over peripheral or cranial nerves.

The first stimulation sequence 406 may be delivered to the patient via one or more first stimulation electrodes (e.g., the stimulation electrodes 120) positioned on the cranium and/or scalp of the patient. The one or more first stimulation electrodes may include one, two, three, four, five, or more stimulation electrodes. One or more (e.g., one, two, three, four, five, or more) recording electrodes, such as the recording electrodes 110 may record one or more electrical waveforms in response to the delivery of the first stimulation sequence 406.

The first stimulation sequence 406 may be used to acquire an MEP and/or condition or prime one or more areas of the scalp or brain of the patient for obtaining the MEP. The first stimulation sequence 406 may include one or more (e.g., a plurality, one, two, three, four, five, or more) first stimulation pulses 430. The one or more first stimulation pulses 430 may be delivered to one or more areas of the scalp or brain of the patient at, for example, the cranium or scalp of the patient. In some implementations, the one or more first stimulation pulses 430 defines a first stimulation train. Thus, a train length 404 of the first stimulation sequence 406 may be one, two, three, four, five, or more pulses.

Referring to FIG. 4, the third stimulation sequence 408 may be used to acquire a second MEP. The third stimulation sequence 408 may include one or more (e.g., a plurality, one, two, three, four, five, or more) third stimulation pulses 434. The one or more third stimulation pulses 434 may be delivered to one or more areas of the patient at, for example, the cranium or scalp of the patient. In some implementations, the one or more third stimulation pulses 434 defines a third stimulation train. Thus, a train length of the third stimulation sequence 408 may be one, two, three, four, five, or more pulses.

In some implementations, each pulse of the one or more first stimulation pulses 430 and the one or more third stimulation pulses 434 may include one or more pulse characteristics such as an amplitude or pulse intensity 410, a duration 412 or pulse width, an inter-stimulus interval (ISI) 414, a frequency, a pulse train period 418 (e.g., a summation of the ISI 414 and the stimulus duration 412), and/or the like. In some implementations, each pulse of the one or more first stimulation pulses 430 has the same pulse characteristics. In some implementations, one or more pulses of the one or more first stimulation pulses 430 and the one or more third stimulation pulses 434 has one or more different characteristics. In some implementations, one or more of the pulse characteristics is ramped compared to a previous pulse in the first stimulation train and/or the third stimulation train. For example, in some implementations, the pulse intensity 410, the duration 412, the ISI 414, and/or the frequency of a subsequent pulse may increase compared to a prior pulse in the first stimulation train and/or third stimulation train. Additionally, or alternatively, in some implementations, the pulse intensity 410, the duration 412, the ISI 414, and/or the frequency of a subsequent pulse may decrease compared to a prior pulse in the first stimulation train and/or the third stimulation train. The one or more pulse characteristics of the stimulation pulses of the first stimulation train and/or third stimulation train may be predetermined and/or dynamically adjusted by system 100 automatically or by a user operating system 100. The one or more pulse characteristics of the stimulation pulses of the first stimulation train and/or the third stimulation train—whether predetermined or dynamically adjusted—may be affected or dictated by available patient data, such as SSEP data, recently acquired MEP data (whether facilitated or not), and/or the like.

In some implementations, the pulse intensity 410 of the stimulation pulses of the first stimulation sequence 406 may be approximately 25 to 1000 volts, 1 to 25 volts, 25 to 50 volts, 50 to 100 volts, 100 to 250 volts, 250 to 500 volts, 500 to 750 volts, 750 to 1000 volts, 1000 to 1250 volts, or more or other ranges therebetween. In some implementations, the stimulus duration 412 is approximately 50 to 500 μsec., 1 to 50 μsec., 50 to 100 μsec., 100 to 200 μsec., 200 to 300 μsec., 300 to 400 μsec., 400 to 500 μsec., 500 to 600 μsec., or greater, or other ranges therebetween. In some implementations, the ISI 414 is approximately 1 to 5 ms, 0.25 to 1 ms, 1 to 2 ms, 2 to 3 ms, 3 to 4 ms, 4 to 5 ms, 5 to 6 ms, 6 to 7 ms, or greater or other ranges therebetween. The ISI 414 may be adjusted in 0.25 ms increments.

The third stimulation sequence 408 may be delivered to the patient after the first stimulation sequence 406 and/or the second stimulation sequence 407. The third stimulation sequence 408 may be delivered to the patient after the first stimulation sequence 406 and temporally spaced apart from the first stimulation sequence 406 by an inter-train interval (“ITI”) 416. The ITI 416 may be approximately 10 to 500 ms. In some implementations the ITI 416 is approximately 15 to 40 ms or 200 to 500 ms. In some implementations, the ITI 416 is approximately 10 to 50 ms, 50 to 100 ms, 100 to 200 ms, 200 to 300 ms, 300 to 400 ms, 400 to 500 ms, 500 to 600 ms, 600 to 700 ms, or greater, or other ranges therebetween. The ITI 416 may be predetermined and/or dynamically adjusted, for example in 1 ms, 2 ms, 5 ms, 10 ms, 15 ms, 20 ms, or 50 ms increments.

In some implementations, the second stimulation sequence 407 is the first stimulation sequence delivered as part of an MEP acquisition procedure. In such a procedure, then, the ITI 416 is irrelevant or essentially non-existent. In some implementations, the second stimulation sequence 407 is the first stimulation sequence delivered as part of an MEP acquisition procedure performed after an earlier MEP acquisition procedure. Accordingly, the ITI 416 in such a situation may be between approximately 1 minute to 240 minutes, between approximately 2 minutes and 20 minutes, between approximately 3 minutes and 15 minutes, and/or any time range therebetween.

As noted above, the first stimulation sequence 406 may prime or condition one or more neural pathways of the patient for acquisition of the MEP by delivering the third stimulation sequence 408. Thus, when the first stimulation sequence 406 is paired with the third stimulation sequence 408, the first stimulation sequence 406 may define a conditioning stimulation train. As the first stimulation sequence 406 may define the conditioning train, the first stimulation sequence 406 may include fewer pulses (e.g., two to three pulses) and/or a lower intensity (e.g., 150 to 250 volts) compared to the stimulation pulses of the third stimulation sequence 408. In such implementations, the third stimulation sequence 408 may define the stimulation train. Thus, the third stimulation sequence 408 may include a greater number of stimulation pulses (e.g., four to six pulses), and/or a higher intensity (e.g., 200 to 300 volts) compared to the first stimulation sequence 406. In some implementations, a fourth stimulation sequence may be delivered, such as if the third stimulation sequence 408 does not produce an MEP or a suitable MEP.

Referring again to FIG. 4, the stimulation sequence 400 may include the second stimulation sequence 407. As noted above, the second stimulation sequence 407 may be delivered to one or more peripheral nerves of the patient either simultaneously or in quick succession. The second stimulation sequence 407 may be delivered from an electrode otherwise used to acquire an SSEP and/or from a separate stimulation electrode used only to facilitate the acquisition of the MEP. In other words, delivery of the second stimulation sequence 407 may improve acquisition of the MEP via the third stimulation sequence 408. The second stimulation sequence 407 may include one or more (e.g., a plurality, one, two, three, four, five, or more) second stimulation pulses 422. In some implementations, the second stimulation sequence 407 includes a single stimulation pulse 422, such as a single peripheral facilitation pulse. The one or more second stimulation pulses 422 may be delivered to one or more peripheral nerves of the patient at, for example, one or more of limbs, the neck, and/or extremities of the patient either simultaneously or sequentially.

The stimulation pulse 422 may have a stimulus duration of approximately 300 μsec. The stimulation pulse 422—which may comprise one, two, three, or more stimulations—may have a stimulus duration of approximately 50 to 100 μsec., 100 to 150 μsec., 150 to 200 μsec., 200 to 250 μsec., 250 to 300 μsec., 300 to 350 μsec., 350 to 400 μsec., 400 to 450 μsec., greater, and/or other ranges therebetween. The second stimulation sequence 407 including the stimulation pulse 422 may be delivered to the patient (e.g., automatically, manually initiated, and/or the like) during the ITI 407. For example, the second stimulation sequence 407 may be delivered between the first stimulation sequence 406 and the third stimulation sequence 408.

The second stimulation sequence 407 may be delivered to the patient (e.g., automatically, manually initiated, and/or the like) prior to the third stimulation sequence 408 by a peripheral to central inter-stimulus interval (“PCISI”) 420. The PCISI 420 may be approximately 56 to 64 ms. The PCISI 420 may be approximately 10 to 30 ms. The PCISI 420 may be approximately 90 to 150 ms. The PCISI 420 may be approximately 10 to 50 ms, 50 to 100 ms, 100 to 150 ms, 150 to 200 ms, lesser, greater, or other ranges therebetween. Thus, the second stimulation sequence 407 may facilitate the acquisition of the MEP by delivering the stimulation to the one or more peripheral nerves of the patient prior to the delivery of stimulation to the one or more cranial nerves of the patient.

In particular, the second stimulation sequence 407 may be delivered to a region of interest of the patient. The region of interest may be a portion of the body of the patient, such as the limb, arm, leg, foot, hand, face and/or the like, of the patient. The region of interest may be the region of the patient that is at-risk during the surgery, such as the spinal surgery. Thus, during surgery, it can be beneficial to determine whether the patient has motor function in the region of interest. The region of interest may be a peripheral region of the patient. In some implementations, delivering the second stimulation sequence 407 to the region of interest prior to delivering the third stimulation sequence 408 to the cranium of the patient may help to limit movement, in response to the delivery of the third stimulation sequence 408, of the patient to the region of interest of the patient, thereby reducing disruptions in the surgical procedure and allowing for more continuous collection of data during the surgical procedure.

Additionally and/or alternatively, delivering the second stimulation sequence 407 to the region of interest prior to delivering the third stimulation sequence 408 to the cranium of the patient may allow for a reduction in the voltage or pulse intensity of at least one of the pulses of the third stimulation sequence 408 (e.g., one, two, three, four, five, six, or more or all of the pulses). Additionally, and/or alternatively, delivering the second stimulation sequence 407 to the region of interest prior to delivering the third stimulation sequence 408 to the cranium of the patient may reduce the likelihood of a false positive and/or false negative detection of a motor or physiological response.

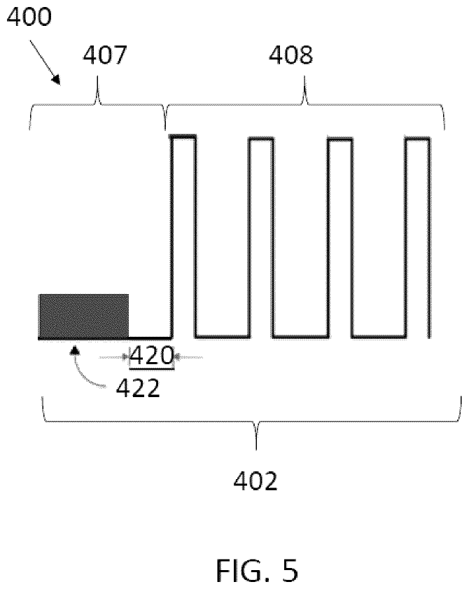

FIG. 5 illustrates an example of the stimulation sequence 400, consistent with implementations of the current subject matter. For example, as shown in FIG. 5, the stimulation sequence may not include the first stimulation sequence 406. In other words, the stimulation sequence 400 may include only the second stimulation sequence 407 (in this case, the first stimulation sequence of the stimulation sequence 400) and the third stimulation sequence 408 (in this case, the second stimulation sequence of the stimulation sequence 400). Additionally, and/or alternatively, the stimulation sequence 400 shows an example stimulation sequence that includes the second stimulation sequence 407 being delivered to one or more peripheral or cranial nerves of the patient, such as at the region of interest, prior to delivering the third stimulation sequence 408 to one or more areas of the scalp or cranium of the patient.

In some implementations, a method of performing surgery or other procedure as described herein includes performing a robotically-assisted surgical procedure, such as, for example, a robotically-assisted hysterectomy, other gynecologic surgical procedure, prostatectomy, urologic surgical procedure, general laparoscopic surgical procedure, thoracoscopic surgical procedure, valve replacement, other cardiac surgical procedure, bariatric surgery, other gastrointestinal surgical procedure, or oncological surgical procedures, among others. The method of some implementations further includes delivering an electrical stimulus to a peripheral or cranial nerve in the body, recording a resultant electrical waveform generated by the body's nervous system in response to the electrical stimulus, and monitoring the resultant electrical waveform to detect changes indicative of potential nerve injury. The method of some implementations further includes delivering an electrical stimulus to the scalp or cranium or brain, recording a resultant electrical waveform generated by the body's nervous system in response to the electrical stimulus, and monitoring the resultant electrical waveform to detect changes indicative of potential nerve injury or motor functioning. Additionally, or alternatively, in some implementations, the method of performing surgery may include any of the methods for detecting the functionality of one or more nerves or nerve tracts described elsewhere herein. The methods of detecting functionality of one or more nerves or of using the response identification device 101 may be incorporated at any juncture of a robotic surgery. For example, such methods can be performed at multiple times, continuously, at pre-selected situations such as when certain types of procedures are initiated or concluded (including any of those mentioned above), and so forth. The method of various implementations further includes adjusting the position of a patient when a potential nerve injury or abnormality is detected.

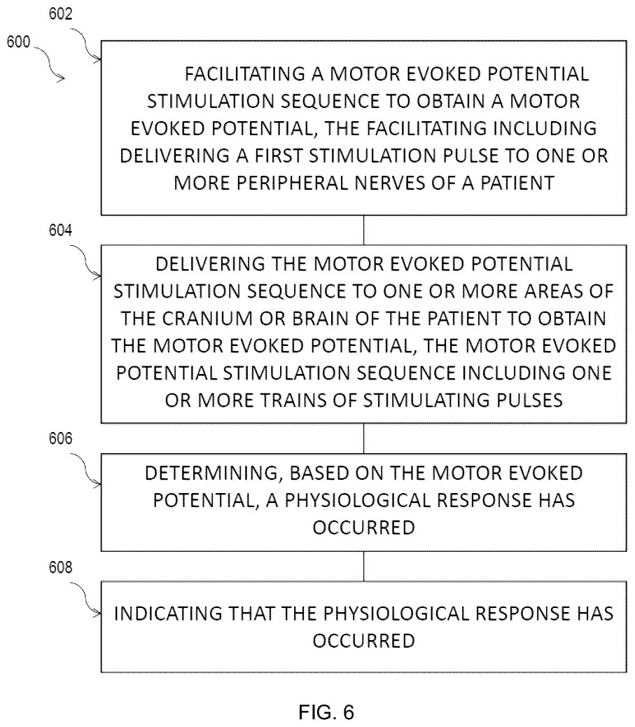

FIGS. 6-8 depict methods 600, 700, 800, respectively, implemented by the stimulation system 100 described herein. One or more of the steps of the method 600 may be implemented in the method 700 and/or the method 800. One or more of the steps of the method 700 may be implemented in the method 600 and/or the method 800. One or more of the steps of the method 800 may be implemented in the method 700 and/or the method 600.

FIG. 6 depicts a method 600 for facilitating an MEP stimulation sequence to obtain an MEP, consistent with implementations of the current subject matter.

At 602, the system (e.g., via the identification controller 102) may facilitate an MEP stimulation sequence used to obtain an MEP. Facilitation of the MEP stimulation sequence may include delivering, via a first stimulation electrode, a first stimulation pulse to one or more peripheral nerves of a patient. The one or more peripheral nerves of the patient may include the brachial plexus, peroneal nerve, femoral nerve, lateral femoral cutaneous nerve, sciatic nerve, spinal accessory nerve, tibial nerve, one or more nerves of the somatic nervous system, one or more nerves of the autonomic nervous system, and/or the like. The one or more peripheral nerves may be positioned at least partially within a region of interest. In some implementations, the first stimulation electrode is coupled to the patient in the region of interest. The region of interest may be a portion of the body of the patient, such as a limb, arm, leg, foot, hand, ankle, wrist, fingers, face and/or the like, of the patient.

In some implementations, the first stimulation pulse includes a single stimulation pulse. In some implementations, the first stimulation pulse includes a plurality of stimulation pulses. In some implementations, the first stimulation pulse may be delivered to the one or more peripheral nerves of the patient by the same electrode used to obtain an SSEP. When collected, the SSEP may indicate that the surgeon is approaching a nerve or nerve structure during the surgical procedure, such as the spinal surgical procedure. The one or more peripheral nerves may include a tibial nerve (e.g., posterior tibial nerve), a saphenous nerve, an ulnar nerve, and/or the like.

In some implementations, the SSEP stimulation system described herein facilitates the MEP stimulation sequence by sharing one or more SSEPs stimulation sites or stimulation pulses and by correlating changes in the two stimulation techniques.

At 604, the system (e.g., via the identification controller 102) may deliver, after the facilitating and via a second stimulation electrode, the MEP stimulation sequence to one or more peripheral or cranial nerves of the patient to obtain the MEP. The MEP stimulation sequence may include a train of stimulation pulses. In some implementations, the second stimulation electrode is coupled to a scalp or cranium of the patient. In some implementations, the MEP acquisition system described herein may deliver the MEP stimulation sequence.

In some implementations, prior to the facilitating, and via a second stimulation electrode, the system (e.g., via the identification controller 102) may deliver a second MEP stimulation sequence to the one or more cranial or scalp areas of the patient. The second MEP stimulation sequence may include a second train of stimulation pulses. The second MEP stimulation sequence may be delivered prior to the MEP stimulation sequence. For example, the second MEP stimulation sequence may be delivered prior to the MEP stimulation sequence and spaced from the MEP stimulation sequence by an inter-train time interval. The inter-train time interval may be approximately 10 to 500 ms. In some implementations the inter-train time interval 416 is approximately 15 to 40 ms or 200 to 500 ms. In some implementations, the inter-train time interval 416 is approximately 10 to 50 ms, 50 to 100 ms, 100 to 200 ms, 200 to 300 ms, 300 to 400 ms, 400 to 500 ms, 500 to 600 ms, 600 to 700 ms, or greater, or other ranges therebetween.

In some implementations, the facilitating occurs during the inter-train time interval. For example, the facilitating may occur within a period of time before the MEP stimulation sequence and/or after the first MEP stimulation sequence. In other words, the MEP stimulation sequence may be delivered within the period of time after the conditioning of the MEP stimulation sequence.

In some implementations, the period of time is predetermined. In some implementations, the period of time is dynamically adjusted based on one or more parameters of the patient and/or a type of surgery being performed on the patient. For example, the one or more parameters of the patient may include the age, height, weight, gender, health, and/or the like of the patient. Additional parameters that may be used to adjust one more parameters of the MEP stimulation/acquisition procedure include the results of SSEP monitoring of the patient, the results of EMG monitoring of the patient, and the results of prior MEP monitoring of the patient.

In some implementations, the facilitating occurs automatically at a particular time after a first or second MEP conditioning stimulation sequence and/or prior to delivering the MEP stimulation sequence. For example, after the second MEP conditioning stimulation sequence is delivered to the patient, the first stimulation pulse may be delivered to the patient at a particular time (or within a period of time) after the second MEP conditioning stimulation sequence. Additionally and/or alternatively, the first stimulation pulse may be automatically delivered to the patient at a particular time (or within a period of time) prior to delivery of the MEP stimulation sequence. Additionally and/or alternatively, the MEP stimulation sequence may be delivered automatically at a particular time (or within a period of time) after delivery of the stimulation pulse as part of the facilitation. Automatically facilitating the MEP stimulation sequence and/or delivering the MEP stimulation sequence may help the surgeon during surgery by reducing the need for a technologist in the operating room, by reducing disruptions during the surgical procedure, by reducing patient movement, by reducing the length of the surgical procedure, and/or the like. In some implementations, the facilitating (e.g., the delivery of the stimulation pulse to a peripheral or cranial nerve), the entire MEP stimulation sequence, and/or the second MEP stimulation sequence is initiated manually, such as at the response identification device 101.

At 606, the system (e.g., via the identification controller 102) may determine, based on the MEP, if a physiological response has occurred. For example, the system may determine that the physiological response has occurred by comparing an initial waveform to an expected baseline waveform in one or more of shape, size or morphology, by algorithmic classification or other means or in comparing one or more waveforms of the MEP collected after baselines are collected to a baseline waveform. In some implementations, the system determines that the physiological response has occurred by comparing one or more features, such as but not limited to an amplitude, a waveform shape, a frequency, a latency, and/or the like, of the MEP to a threshold. In such implementations, the system may detect a presence of the physiological response when the one or more features is greater than or equal to the threshold. Additionally or alternatively, the system may detect a presence of the physiological response when the one or more features is less than or equal to the threshold.

At 608, the system (e.g., via the identification controller 102) may indicate, via a display (e.g., the display 54) coupled to the first stimulation electrode and the second stimulation electrode, that the physiological response has occurred. The display may be coupled to the response identification device 101, the SSEP acquisition system 104, the MEP acquisition system 106, and/or other components of the stimulation system 100. The indication may include one or more alerts, such as one or more audio, visual, and/or tactile alerts or signals. The indication may indicate that the surgeon is approaching or interfering with a nerve or nerve tract of a patient. The indication may indicate that the patient has motor function in the region of interest. In some implementations, the indication indicates that no physiological response has occurred or that the response has changed. In such instances, the surgeon may need to pause surgery, wake up the patient, and/or alter the surgical procedure.

Thus, the stimulation system, such as by facilitating the MEP stimulation sequence, may reduce an intensity of the pulses, the number of pulses per train or the number of trains of stimulation pulses of the MEP stimulation sequence, limit movement of the patient to the region of interest during the delivery of the MEP stimulation, and/or improve an accuracy of determining that the physiological response has occurred or changed.

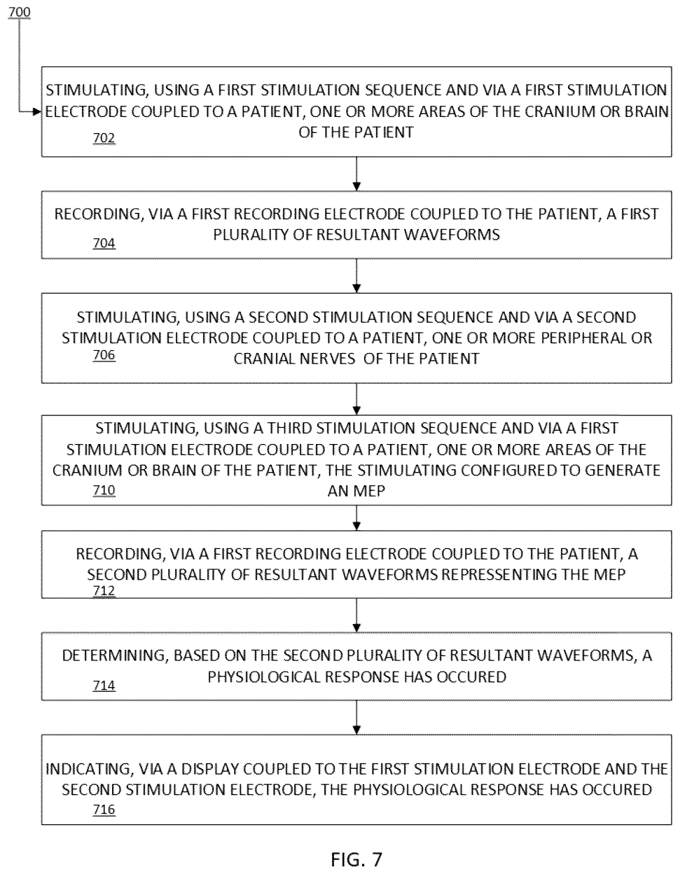

FIG. 7 depicts a method 700 for facilitating an MEP stimulation sequence to obtain an MEP, consistent with implementations of the current subject matter.

At 706, the system, such as the system 100 (e.g., via the identification controller 102) may stimulate, using a second stimulation sequence and via a first stimulation electrode coupled to a patient, one or more peripheral nerves of the patient. The first stimulation electrode may include one, two, three, four, five, six, seven, eight or more stimulation electrodes. The first stimulation electrode may be positioned on a region of interest of the patient. For example, during various types of surgery it can be desirable to determine whether the patient has movement in their limbs, feet, legs, hands, wrist, and/or the like. The region of interest may include the region of the patient, such as the limbs, feet, legs, hands, wrist, face and/or the like. The region of interest may be the region of the patient that is at-risk during the surgery, whether that surgery be a spinal surgery or any other surgery where nerve health is a priority or at risk or where the status of the patient's nerve health may be relevant to the procedure. For example, during surgery, it can be beneficial to determine whether the patient has motor functioning in the region of interest. The region of interest may be a peripheral region of the patient.

At 702, the system, such as the system 100 (e.g., via the identification controller 102) may stimulate, using a first stimulation sequence and via a second stimulation electrode coupled to a patient, one or more areas of the scalp, cranium or brain of the patient. The second stimulation electrode may include one, two, three, four, five, six, seven, eight or more stimulation electrodes. The second stimulation electrode may be positioned over one or more areas of the scalp, cranium or brain of the patient. In some implementations, the stimulation sequence includes a first plurality of stimulation pulses as part of a first train of stimulation pulses, as described herein. One or more of the stimulation pulses of the first plurality of stimulation pulses may include the same amplitude, the same pulse width, and/or the same frequency. Additionally and/or alternatively, one or more of the stimulation pulses of the first plurality of stimulation pulses may include a different amplitude, pulse width, and/or frequency. The first plurality of stimulation pulses may include one, two, three, four, five, six, or more stimulation pulses. The first stimulation pulse may prime the brain for obtaining the MEP. Priming or conditioning the brain in combination with facilitating the MEP stimulation sequence may help to produce more accurate results and may help to reduce the likelihood of injury caused to the patient and/or reduce the likelihood that the surgery will be significantly disrupted. Additionally and/or alternatively, the priming or conditioning may occur before or simultaneously with the facilitating at 706. Additionally and/or alternatively the first stimulation sequence may not be delivered.

At 704, the system 100, such as via the identification controller 102, may record, via a first recording electrode coupled to the patient, a first plurality of resultant electrical waveforms. The first plurality of resultant electrical waveforms may include one, two, three, four, five, or more resultant electrical waveforms. The first plurality of resultant electrical waveforms may be received by the response identification device. The resultant electrical waveforms may be generated by the patient in response to the delivered electrical stimuli, such as the first stimulating sequence. In some implementations, the first stimulation electrode and/or the first recording electrode may form a part of the MEP acquisition system 106.

In some implementations, the second stimulation sequence follows the first stimulation sequence or occurs during it and facilitates a third stimulation sequence. In other words, the second stimulation sequence may be applied to the region of interest of the patient prior to delivering the third stimulation sequence to limit the patient physiological response to the third stimulation sequence to the region of interest. In some implementations, the second stimulation electrode and/or the second recording electrode (described in more detail below) may form a part of the SSEP acquisition system 104.

In some implementations, the second stimulation sequence includes a single stimulation pulse. In some implementations, the second stimulation sequence includes a plurality of stimulation pulses as part of a second stimulation train, as described herein. In some implementations, such as when the second stimulation sequence includes a second plurality of stimulation pulses, one or more of the stimulation pulses of the second plurality of stimulation pulses may include the pulses of the same amplitude, the same pulse width, and/or the same frequency. Additionally and/or alternatively, one or more of the stimulation pulses of the second plurality of stimulation pulses may include a different amplitude, pulse width, and/or frequency.

At 710, the system, such as the system 100 (e.g., via the identification controller 102) may stimulate, using a third stimulation sequence and via the first stimulation electrode coupled to the patient, the one or more cranial nerves of the patient. In some implementations, the third stimulation sequence is the same as the first stimulation sequence. In some implementations, the third stimulation sequence is different from the first stimulation sequence. In some implementations, the third stimulation sequence includes a third plurality of stimulation pulses as part of a third train of stimulation pulses, as described herein. One or more of the stimulation pulses of the third plurality of stimulation pulses may have the same amplitude, the same pulse width, and/or the same frequency. Additionally and/or alternatively, one or more of the stimulation pulses of the third plurality of stimulation pulses may have a different amplitude, pulse width, and/or frequency. The third plurality of stimulation pulses may include one, two, three, four, five, six, or more stimulation pulses. The third plurality of stimulation pulses obtain the MEP.

At 712, the system 100, such as via the identification controller 102, may record, via a second recording electrode coupled to the patient, a second plurality of resultant electrical waveforms. The second plurality of resultant electrical waveforms may include one, two, three, four, five, or more resultant electrical waveforms. The second plurality of resultant electrical waveforms may be received by the response identification device. The second plurality of resultant electrical waveforms may be generated by the patient in response to the delivered electrical stimuli, such as the second stimulating sequence.

In some implementations, stimulating using the second stimulation sequence occurs during an inter-train time interval between the stimulating using the first stimulation sequence and the stimulating using the third stimulation sequence. For example, the second stimulation sequence may occur within a period of time, such as the PCISI 420 before the third stimulation sequence and/or after the first stimulation sequence. In other words, the third stimulation sequence may be delivered within the period of time after the delivery of the second stimulation sequence.

In some implementations, stimulating using the second stimulation sequence occurs prior to or during the stimulating using the first stimulation sequence and the stimulating using the third stimulation sequence. For example, the second stimulation sequence may occur within a period of time, such as the PCISI 420 before the third stimulation sequence and/or either before or after the first stimulation sequence. In other words, the third stimulation sequence may be delivered within the period of time after the delivery of the second stimulation sequence.

In some implementations, the period of time is predetermined. In some implementations, the period of time is dynamically adjusted based on one or more parameters of the patient and/or a type of surgery being performed on the patient. For example, the one or more parameters of the patient may include the age, height, weight, gender, health, and/or the like of the patient. The one or more parameters may also include current or past SSEP, MEP, or EMG data.

In some implementations, the second stimulation sequence is delivered automatically at a particular time after the first stimulation sequence and/or prior to delivering the third stimulation sequence. For example, after the first stimulation sequence is delivered to the patient, the second stimulation sequence may be delivered to the patient at a particular time (or within a period of time) after the first stimulation sequence. Additionally and/or alternatively, the second stimulation sequence may be automatically delivered to the patient at a particular time (or within a period of time) prior to delivery of the third stimulation sequence. Additionally and/or alternatively, the third stimulation sequence may be delivered automatically at a particular time (or within a period of time) after delivery of the second stimulation sequence. Automatically delivering the second stimulation sequence and/or delivering the third stimulation sequence may help the surgeon during surgery by reducing the need for a technologist in the operating room, by reducing disruptions during the surgical procedure, by reducing patient movement, by reducing the length of the surgical procedure, and/or the like. In some implementations, the facilitating (e.g., the delivery of the second stimulation sequence), the third stimulation sequence, and/or the first stimulation sequence is initiated manually, such as at the response identification device 101. In some implementations, the first stimulation sequence is not delivered.

At 712, the system 100, such as via the identification controller 102, may record, via the first recording electrode coupled to the patient, a second plurality of resultant electrical waveforms. The second plurality of resultant electrical waveforms may include one, two, three, four, five, or more resultant electrical waveforms. The second plurality of resultant electrical waveforms may be received by the response identification device. The second plurality of resultant electrical waveforms may be generated by the patient in response to the delivered electrical stimuli, such as the third stimulating sequence. In some implementations, one or more of the resultant electrical or mechanical waveforms of the second plurality of resultant electrical or mechanical waveforms represents the MEP.

At 714, the system (e.g., via the identification controller 102) may determine, based on the MEP, if a physiological response has occurred. For example, the system may determine that the physiological response has occurred by comparing one or more resultant electrical waveforms of the MEP to a baseline waveform. In some implementations, the system determines that the physiological response has occurred by comparing one or more features, such as an amplitude, a waveform shape, a frequency, a waveform shape and/or the like, of the MEP to a threshold. In such implementations, the system may detect a presence of the physiological response when the one or more features is greater than or equal to the threshold. Additionally or alternatively, the system may detect a presence of the physiological response when the one or more features is less than or equal to the threshold. Such maximum or minimum threshold may be a latency, a duration, a magnitude, a frequency, a waveform shape or the like.

At 716, the system (e.g., via the identification controller 102) may indicate, via a display (e.g., the display 54) coupled to the first stimulation electrode and the second stimulation electrode, that the physiological response has occurred. The display may be coupled to the response identification device 101, the SSEP acquisition system 104, the MEP acquisition system 106, and/or other components of the stimulation system 100. The indication may include one or more alerts, such as one or more audio, visual, and/or tactile alerts or signals. The indication may indicate that the surgeon is approaching or interfering with a nerve or nerve tract of a patient. The indication may indicate that the patient has motor functioning and/or has feeling in the region of interest. In some implementations, the indication indicates that no physiological response has occurred. In such instances, the surgeon may need to pause surgery, wake up the patient, and/or alter the surgical procedure.

Thus, the stimulation system, such as by facilitating the third stimulation sequence with the second stimulation sequence, may reduce an intensity and/or duration of the train of stimulation pulses of the third stimulation sequence, limit movement of the patient to the region of interest during the delivery of the third stimulation sequence to obtain the MEP, and/or improve an accuracy of determining that the physiological response has occurred.

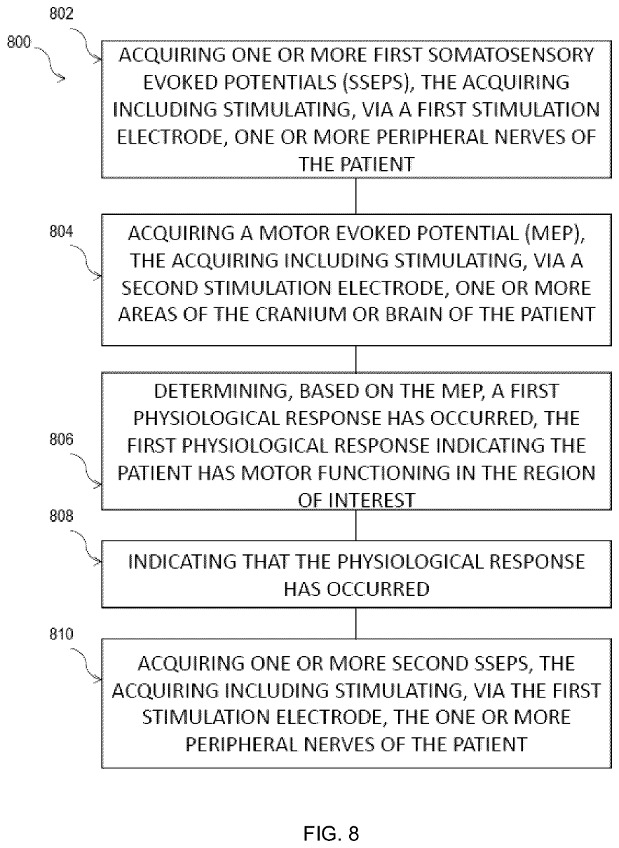

FIG. 8 depicts a method 800 for obtaining MEPs and SSEPs, consistent with implementations of the current subject matter.

At 802, the system (e.g., via the identification controller 102) may acquire one or more first SSEPs. Acquiring the one or more first SSEPs may include stimulating via a first stimulation electrode, one or more peripheral nerves of a patient. The one or more peripheral nerves may be positioned at least partially within a region of interest. In some implementations, the first stimulation electrode is coupled to the patient in the region of interest. The region of interest may be a portion of the body of the patient, such as the limb, arm, leg, foot, hand, and/or the like, of the patient.

In some implementations, the stimulating via the first stimulation electrode includes delivering a stimulation pulse. In some implementations, the stimulation pulse is a single stimulation pulse. In some implementations, the stimulation pulse includes a plurality of stimulation pulses. In some implementations, the stimulation pulse may be delivered to the one or more peripheral nerves of the patient to obtain one or more first SSEPs. The first SSEPs may indicate that the surgeon is approaching or interfering with a nerve or nerve tract during the surgical procedure, such as the spinal surgical procedure. The one or more peripheral nerves may include a tibial nerve (e.g., posterior tibial nerve), a saphenous nerve, an ulnar nerve, and/or the like.

In some implementations, stimulating the one or more peripheral nerves of the patient, as described herein, facilitates the acquisition of an MEP. In doing so, the stimulation to the one or more peripheral nerves of the patient may be delivered to the region of interest of the patient prior to acquiring the MEP to focus the patient physiological response to the stimulation of one or more cranial nerves of the patient to the region of interest.

At 804, the system (e.g., via the identification controller 102) may then acquire an MEP. Acquiring the MEP may include stimulating, via a second stimulation electrode, one or more scalp or cranial areas of the patient to obtain the MEP. The stimulating may include delivering a train of stimulation pulses. In some implementations, the second stimulation electrode is coupled to a scalp of the patient.

In some implementations, the acquisition of the one or more first SSEPs occurs automatically at a particular time prior to acquiring the MEP, and/or the MEP may be acquired automatically at a particular time after the one or more first SSEPs stimulations are delivered. The first stimulation electrode used in acquiring the first SSEPs may also be used for facilitating the acquisition of the MEP.

In some implementations, the acquisition of the one or more first SSEPs and delivery of SSEP stimulating pulses occurs automatically upon the occurrence of a pre-established event, such as the results of ongoing SSEP monitoring of the patient, the results of EMG monitoring of the patient, and/or the results of prior MEP monitoring of the patient. Automatically acquiring the MEP may help the surgeon during surgery by reducing the need for a technologist in the operating room. Utilizing the SSEP stimulating electrode for facilitating the MEP may reduce the number of needed electrodes coupled to the patient. Facilitating the MEP using the one or more of the SSEP stimulating electrodes may further help the surgeon by reducing disruptions during the surgical procedure, by reducing patient movement, by reducing the length of the surgical procedure, and/or the like. In some implementations, the facilitating (in this example, through the use of the SSEP electrodes to deliver the second stimulation sequence prior to the MEP) prior to the acquisition of the MEP is initiated manually, such as at the response identification device 101. In some implementations, the facilitating prior to the acquisition of the MEP is triggered automatically based on one or more parameters, such as the time elapsed from a prior MEP, SSEP, or EMG acquisition, the time elapsed of the surgical procedure, the results of the most recently performed MEP, SSEP, or EMG, the results of one or more of the MEPs, SSEPs, or EMGs performed previously during the surgical procedure, or some the results of another monitoring system, such as a heart rate monitor or the like.

At 806, the system (e.g., via the identification controller 102) may determine, based on the MEP, if a first physiological response has occurred. For example, the system may determine that the first physiological response has occurred by comparing one or more waveforms of the MEP to an expected response or to a baseline waveform. In some implementations, the system determines that the physiological response has occurred by comparing one or more features, such as an amplitude, a waveform shape, a latency, a frequency, and/or the like, of the MEP to a threshold. In such implementations, the system may detect a presence of the physiological response when the one or more features is greater than or equal to the threshold. Additionally or alternatively, the system may detect a presence of the physiological response when the one or more features is less than or equal to the threshold. The first physiological response may indicate that the patient has motor functioning in the region of interest, such as the patient's arm, leg, foot, hand, wrist, limb, face and/or the like.

At 808, the system (e.g., via the identification controller 102) may indicate, via a display (e.g., the display 54) coupled to the first stimulation electrode and the second stimulation electrode, that the physiological response has occurred. The display may be coupled to the response identification device 101, the SSEP acquisition system 104, the MEP acquisition system 106, and/or other components of the stimulation system 100. The indication may include one or more alerts, such as one or more audio, visual, and/or tactile alerts or signals. The indication may indicate that the surgeon is approaching or interfering with a nerve or nerve tract of a patient. The indication may indicate that the patient has motor functioning and/or has feeling in the region of interest. In some implementations, the indication indicates that no physiological response has occurred. In such instances, the surgeon may need to pause surgery, wake up the patient, and/or alter the surgical procedure and/or the patient's position. In some implementations, the system may provide an indication to the surgeon as to what solutions are available and/or recommended based on the results of the MEP acquisition.

At 810, the system (e.g., via the identification controller 102) may acquire one or more second SSEPs. Acquiring the one or more second SSEPs may include stimulating via the first stimulation electrode, the one or more peripheral nerves of a patient. Accordingly, the system described herein may acquire (e.g., automatically acquire) SSEPs and MEPs during a surgical procedure.

Thus, the stimulation system, such as by facilitating the acquisition of MEPs, may reduce an intensity of the stimulation delivered to the one or more scalp or cranial areas during MEP acquisition, limit movement of the patient to the region of interest during MEP acquisition, and/or improve an accuracy of determining that the physiological response has occurred.

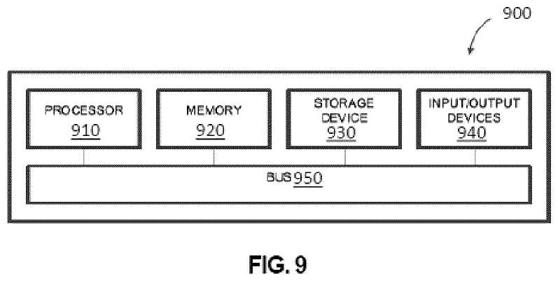

FIG. 9 depicts a block diagram illustrating a computing system 900 consistent with implementations of the current subject matter. Referring to FIGS. 1 and 9, the computing system 900 can be used to implement the stimulation system 100 and/or any components therein.

As shown in FIG. 9, the computing system 900 can include a processor 910, a memory 920, a storage device 930, and input/output devices 940. The processor 910, the memory 920, the storage device 930, and the input/output devices 940 can be interconnected via a system bus 950. The processor 910 is capable of processing instructions for execution within the computing system 900. Such executed instructions can implement one or more components of, for example, the identification controller 102. In some example implementations, the processor 910 can be a single-threaded processor. Alternatively, the processor 910 can be a multi-threaded processor. The processor 910 is capable of processing instructions stored in the memory 920 and/or on the storage device 930 to present graphical information for a user interface provided via the input/output device 940.

The memory 920 is a computer readable medium such as volatile or non-volatile that stores information within the computing system 900. The memory 920 can store data structures representing configuration object databases, for example. The storage device 930 is capable of providing persistent storage for the computing system 900. The storage device 930 can be a floppy disk device, a hard disk device, a solid state drive, an optical disk device, or a tape device, or other suitable persistent storage means. The input/output device 940 provides input/output operations for the computing system 900. In some example implementations, the input/output device 940 includes a keyboard and/or pointing device and/or touch sensitive screen. In various implementations, the input/output device 940 includes a display unit for displaying graphical user interfaces.

According to some example implementations, the input/output device 940 can provide input/output operations for a network device. For example, the input/output device 940 can include Ethernet ports or other networking ports to communicate with one or more wired and/or wireless networks (e.g., a local area network (LAN), a wide area network (WAN), the Internet).

In some example implementations, the computing system 900 can be used to execute various interactive computer software applications that can be used for organization, analysis and/or storage of data in various formats. Alternatively, the computing system 900 can be used to execute software applications. These applications can be used to perform various functionalities, e.g., planning functionalities (e.g., generating, managing, editing of spreadsheet documents, word processing documents, and/or any other objects, etc.), computing functionalities, communications functionalities, etc. The applications can include various add-in functionalities or can be standalone computing products and/or functionalities. Upon activation within the applications, the functionalities can be used to generate the user interface provided via the input/output device 940. The user interface can be generated and presented to a user by the computing system 900 (e.g., on a computer screen monitor, etc.).

One or more aspects or features of the subject matter described herein can be realized in digital electronic circuitry, integrated circuitry, specially designed ASICs, field programmable gate arrays (FPGAs) computer hardware, firmware, software, and/or combinations thereof. These various aspects or features can include implementation in one or more computer programs that are executable and/or interpretable on a programmable system including at least one programmable processor, which can be special or general purpose, coupled to receive data and instructions from, and to transmit data and instructions to, a storage system, at least one input device, and at least one output device. The programmable system or computing system may include clients and servers. A client and server are remote from each other and typically interact through a communication network. The relationship of client and server arises by virtue of computer programs running on the respective computers and having a client-server relationship to each other.

These computer programs, which can also be referred to as programs, software, software applications, applications, components, or code, include machine instructions for a programmable processor, and can be implemented in a high-level procedural and/or object-oriented programming language, and/or in assembly/machine language. As used herein, the term “machine-readable medium” refers to any computer program product, apparatus, and/or device, such as for example magnetic discs, optical disks, memory, and Programmable Logic Devices (PLDs), used to provide machine instructions and/or data to a programmable processor, including a machine-readable medium that receives machine instructions as a machine-readable signal. The term “machine-readable signal” refers to any signal used to provide machine instructions and/or data to a programmable processor. The machine-readable medium can store such machine instructions non-transitorily, such as for example as would a non-transient solid-state memory or a magnetic hard drive or any equivalent storage medium. The machine-readable medium can alternatively or additionally store such machine instructions in a transient manner, such as for example, as would a processor cache or other random-access memory associated with one or more physical processor cores.

To provide for interaction with a user, one or more aspects or features of the subject matter described herein can be implemented on a computer having a display device, such as for example a cathode ray tube (CRT) or a liquid crystal display (LCD) or a light emitting diode (LED) monitor for displaying information to the user and one or more hardware buttons, a keyboard and/or a pointing device, such as for example a mouse or a trackball, by which the user may provide input to the computer. Other kinds of devices can be used to provide for interaction with a user as well. For example, feedback provided to the user can be any form of sensory feedback, such as for example visual feedback, auditory feedback, or tactile feedback; and input from the user may be received in any form, including acoustic, speech, or tactile input. Other possible input devices include touch screens or other touch-sensitive devices such as single or multi-point resistive or capacitive track pads, voice recognition hardware and software, optical scanners, optical pointers, digital image capture devices, hardware buttons, and associated interpretation software, and the like.

### Embodiments

The following embodiments are included in this disclosure as non-limiting examples of various combinations of features contemplated by the authors of this disclosure:

Embodiment 1: An automated MEP device configured to optimize one or more of stimulation, acquisition, and analysis parameters for the collection of both facilitated and unfacilitated MEPs, the device comprising:
at least one first stimulating electrode pair placed on or near a peripheral or cranial nerve of a patient;
at least one first recording electrode pair placed on or near the cranium of the patient;
at least one second stimulating electrode pair placed on or near a cranium of the patient;
at least one second recording electrode pair placed on a near a region of interest of the patient;
at least one processor;
at least one memory storing instructions that, when executed by the at least one processor, result in operations that include at least two or more of the following performed one or more times:
   delivering, via the second stimulation electrode pair, a first stimulation sequence comprising one or more electrical pulses
   delivering, via the first stimulating electrode pair, a second stimulation sequence comprising one or more electrical pulses either before, during or after the first stimulation sequence;
   delivering, via the second stimulation electrode pair, a third stimulation sequence comprising one or more electrical pulses; and
   recording, via the second recording electrode pair, an evoked potential;

wherein the processor automatically implements the instructions based on at least one of a predetermined interval, a predetermined delay, the occurrence of a monitored event, the results of a prior implementation of the instructions, and personal characteristics of the patient.

Embodiment 2: The automated device of Embodiment 1, wherein the processor analyzes the evoked potential to optimize one or more of the following of the first and/or second and/or third stimulation sequence: number of pulses, frequency of pulses, pulse amplitude, pulse duration, and duration between the first and second stimulation sequences, the duration between the second and third stimulation sequences and stores the results of the analysis in the memory.

Embodiment 3: The automated device of Embodiment 2, wherein the processor outputs the analysis on a display.

Embodiment 4: The automated device of Embodiment 2 or 3, wherein the processor automatically adjusts one or more parameters of subsequent deliveries of the first and/or second and or third stimulation sequences based on the analysis.

Embodiment 5: The automated device of Embodiment 1, 2, 3, or 4, wherein the device is configured to:
deliver at least one third stimulation sequence not accompanied by a second stimulation sequence (“unfacilitated MEP”) and at least one first, second and third stimulation sequence (conditioned facilitated MEP″) and at least one second stimulation sequence immediately followed by a third stimulation sequence (“facilitated MEP”);
compare the one or more evoked potentials resulting from the unfacilitated MEP to the one or more evoked potentials resulting from the facilitated MEP;
compare the one or more evoked potentials resulting from the facilitated MEP to the evoked potentials resulting from the conditioned facilitated MEP;
compare the one or more evoked potentials resulting from the unfacilitated MEP to the conditioned facilitated MEP;
compare one or more first evoked potentials resulting from an unfacilitated, a facilitated or a conditioned facilitated of evoked potentials to one or more second evoked potentials of the same type;
analyze the comparison to determine at least one of an optimal stimulation pattern, a nerve or nerve tract health of the patient, one or more optimized parameters of the first and/or second and/or third stimulation sequences, a recommendation for adjusting one or more parameters of the first and/or second stimulation sequences, a recommendation for changing a position of the patient, and a recommendation for a change in a surgical operation being performed on the patient; and
store the recommendation in the memory and/or output the recommendation to a display.

Embodiment 6: The automated device of Embodiment 1, 2, 3, 4, or 5, wherein the processor, based on the recorded first and second evoked potentials, determines an optimal amplitude for the first stimulation sequence that will maximize a desired aspect of the second evoked potential.

Embodiment 7: The automated device of Embodiment 6, wherein the desired aspect of the second evoked potential is at least one of a response from a muscle or myotome of interest, a response from a muscle or myotome of interest at or above a threshold, a response at or above a threshold.

Embodiment 8: The automated device of Embodiment 1, 2, 3, 4, 5, 6, or 7, wherein the processor, based on the recorded first and/or second evoked potentials, determines optimal parameters for the second stimulation sequence that maximizes the evoked potential.

Embodiment 9: The automated device of Embodiment 1, 2, 3, 4, 5, 6, 7, or 8, wherein the processor, based on the recorded first and second evoked potentials, determines an optimal time period between the first and second stimulation sequences that maximizes an amplitude of the second evoked potential.

Embodiment 10: The automated device of Embodiment 9, wherein the maximized amplitude is in a muscle or myotome of interest to a user.

Embodiment 11: The automated device of Embodiment 1, 2, 3, 4, 5, 6, 7, 8, 9, or 10, wherein the processor, based on the recorded first and second evoked potentials, determines optimized parameters for the first and second and third stimulation sequences and stores in the memory one of the corresponding second evoked potentials as a baseline.

Embodiment 12: The automated device of Embodiment 10, wherein the device delivers subsequent first and/or second stimulation sequences followed by third stimulation sequences, and the processor compares the resulting evoked potentials to the baseline, the comparison being based on but not limited to one or more of latency, amplitude, morphology, area under the curve and waveform shape.

Embodiment 13: The automated device of Embodiment 12, wherein the device generates an alert if the comparison of the resulting evoked potentials and the baseline exceeds a threshold, wherein the threshold is either predetermined by a user or established by the processor based on published standards or stored data of prior evoked potentials.

Embodiment 14: The automated device of Embodiment 1, 2, 3, 4, 5, 6, 7, 8, 9, 10, 11, 12, or 13, wherein the occurrence of a monitored event is derived from at least one of SSEP data and EMG data, facilitated MEPs, and non-facilitated MEPs obtained from the patient in real time.

Embodiment 15: The automated device of Embodiment 1, 2, 3, 4, 5, 6, 7, 8, 9, 10, 11, 12, 13, or 14, wherein the personal characteristics of the patient is at least one of the patient's height, age, gender, an SSEP latency, and a facilitated, conditioned and facilitated or non-facilitated MEP latency.

Embodiment 16: The automated device of Embodiment 1, 2, 3, 4, 5, 6, 7, 8, 9, 10, 11, 12, 13, 14, or 15, wherein the processor calculates alerting criteria for facilitated MEPs according to one or more factors including but not limited to a MEP amplitude, morphology, area under the curve, a stimulation intensity of the first and/or third stimulation sequences, stimulation sequence, and stimulation pulse duration of the first and/or third stimulation sequences and a trend of one or more of those factors.

Embodiment 17: The automated device of Embodiment 1, 2, 3, 4, 5, 6, 7, 8, 9, 10, 11, 12, 13, 14, 15, or 16, wherein the processor is configured to determine and inform a user of an optimal location on the patient for placement of the at least one first stimulating electrode pair based on the type of procedure being performed on the patient.

Embodiment 18: The automated device of Embodiment 1, 2, 3, 4, 5, 6, 7, 8, 9, 10, 11, 12, 13, 14, 15, 16, or 17, wherein the processor determines when to not implement the operations during a specified period so that no stimulation sequence is delivered to the patient during the specified period, wherein the determination to not implement is informed by an ongoing triggered EMG test being performed on the patient during at least a portion of the specified period.

Embodiment 19: A method of providing neural monitoring of a patient, the method comprising:
providing an automated device according to Embodiment 1, 2, 3, 4, 5, 6, 7, 8, 9, 10, 11, 12, 13, 14, 15, 16, 17, or 18;
affixing the at least one first stimulating electrode pair near a peripheral or cranial nerve of the patient, the peripheral or cranial nerve being located in a region of interest of the patient;
affixing the at least one first recording electrode pair on or near the cranium of the patient;
affixing the at least one second stimulating electrode pair on or near the cranium of the patient; and
affixing the at least one second recording electrode pair near a region of interest of the patient.

Embodiment 20: A stimulation system for detecting and identifying a patient physiological response, the stimulation system comprising:
at least one processor; and
at least one memory storing instructions which, when executed by the at least one processor, result in operations comprising:
   facilitating a motor evoked potential (MEP) stimulation sequence to obtain an MEP, the facilitating comprising delivering, via a first stimulation electrode, a first stimulation pulse to one or more peripheral or cranial nerves of a patient, the one or more peripheral or cranial nerves located within a region of interest, the first stimulation electrode coupled to the patient in the region of interest;
   delivering, after the facilitating and via a second stimulation electrode, the MEP stimulation sequence to one or more areas of the of the patient to obtain the MEP response, the MEP stimulation sequence comprising a train of stimulation pulses, the second stimulation electrode coupled to a scalp of the patient;
   determining, based on the MEP, whether a physiological response has occurred; and
   indicating, via a display coupled to the first stimulation electrode and the second stimulation electrode, that the physiological response has occurred;
   wherein the facilitating is configured to one or more of: optimizing an intensity or pulse duration or number of pulses of the train of stimulation pulses of the MEP stimulation sequence, limit movement of the patient to the region of interest during the delivery of the MEP stimulation, and improve an accuracy of determining whether the physiological response has occurred.

Embodiment 21: The system of Embodiment 20, wherein the first stimulation pulse comprises a single stimulation pulse.

Embodiment 22: The system of Embodiment 20 or 21, wherein delivering the first stimulation pulse to the one or more peripheral nerves of the patient is configured be delivered by an independent stimulator or one used for SSEP stimulation.

Embodiment 23: The system of any one of Embodiment 20, 21, or 22, wherein the operations further comprise delivering, prior, during or after to the facilitating and via the second stimulation electrode, a second MEP stimulation sequence to the one or more areas of the scalp or cranium of the patient, the second MEP stimulation sequence comprising a second train of stimulation pulses.

Embodiment 24: The system of Embodiment 23, wherein the second MEP stimulation sequence is delivered prior to the MEP stimulation sequence by an inter-train time interval; and wherein the facilitating occurs prior, during, or after the inter-train time interval.

Embodiment 25: The system of any one of Embodiment 21, 21, 23, or 24, wherein the operations further comprise delivering the MEP sequence within a period of time after the facilitation of the MEP stimulation sequence.

Embodiment 26: The system of Embodiment 25, wherein the period of time is a predetermined period of time.

Embodiment 27: The system of Embodiment 25, wherein the period of time is dynamically adjusted based on one or more parameters of the patient and/or a type of surgery being performed on the patient.

Embodiment 28: The system of any one of Embodiment 25, 26, or 27, wherein the facilitating occurs automatically within the period of time prior to the delivering the MEP stimulation sequence.

Embodiment 29: The system of any one of Embodiment 20, 21, 22, 23, 24, 25, 26, 27, or 28, wherein the facilitating is manually initiated prior to the delivering the MEP stimulation sequence.

Embodiment 30: The system of any one of Embodiment 20, 21, 22, 23, 24, 25, 26, 27, 28, or 29, wherein the facilitating is automatically initiated by the system in response to one or more inputs from an SSEP monitoring system, an EMG monitoring system, a heart rate monitoring system, a blood pressure monitoring system, or other patient monitoring system.

Embodiment 31: The system of Embodiment 30, wherein one or more of the SSEP monitoring system, the EMG monitoring system, the heart rate monitoring system, the blood pressure monitoring system, and the other patient monitoring system is contained within or is a part of the stimulation system herein claimed.

Embodiment 32: The system of any one of Embodiment 20, 21, 22, 23, 24, 25, 26, 27, 28, 29, 30, or 31, wherein the MEP comprises one or more waveforms.

Embodiment 33: The system of any one of Embodiment 20, 21, 22, 23, 24, 25, 26, 27, 28, 29, 30, 31, or 32, wherein the processor stores the MEP as a baseline waveform.

Embodiment 34: The system of Embodiment 33, wherein the determining comprises comparing the one or more waveforms of the MEP to the baseline waveform.

Embodiment 35: The system of any one of Embodiment 20, 21, 22, 23, 24, 25, 26, 27, 28, 29, 30, 31, 32, 33, or 34, wherein the determining whether the physiological response has occurred comprises:
comparing one or more features of the MEP to a threshold; and/or
detecting a presence of the physiological response when the one or more features is greater than or equal to the threshold.

Embodiment 36: The system of any one of Embodiment 20, 21, 22, 23, 24, 25, 26, 27, 28, 29, 30, 31, 32, 33, or 34, wherein the determining whether the physiological response has occurred comprises:
comparing one or more features of the MEP to a threshold; and/or
detecting a presence of the physiological response when the one or more features is less than or equal to the threshold.

Embodiment 37: The system of any one of Embodiment 20, 21, 22, 23, 24, 25, 26, 27, 28, 29, 30, 31, 32, 33, 34, 35, or 36, further comprising:
a somatosensory evoked potential (SSEP) stimulation/acquisition system configured to use the same facilitation electrode or electrodes as the MEP stimulation sequence, the SSEP stimulation/acquisition system configured to acquire one or more SSEPs during or prior to delivery of the first stimulation pulse; and
an MEP acquisition system configured to deliver the MEP stimulation sequence.

Embodiment 38: The system of Embodiment 20, further comprising:
the stimulation electrode; and
the recording electrode.

Embodiment 39: The system of any one of Embodiment 20, 21, 22, 23, 24, 25, 26, 27, 28, 29, 30, 31, 32, 33, 34, 35, 36, 37, or 38, wherein the second stimulation electrode comprises four or more electrodes.

Embodiment 40: A facilitated motor evoked potential (MEP) detection system for detecting and identifying a patient physiological response, the stimulation system comprising:
at least one data processor; and
at least one memory storing instructions which, when executed by the at least one data processor, result in operations comprising:
   stimulating, using a first stimulation sequence and via a first stimulation electrode coupled to a patient, one or more areas of the scalp or cranium of the patient, the stimulating configured to generate an MEP;
   recording, via a first recording electrode coupled to the patient, a first plurality of resultant waveforms, the first plurality of resultant waveforms representing an unfacilitated MEP;
   stimulating, using a second stimulation sequence and via a second stimulation electrode coupled to the patient, one or more peripheral or cranial nerves of the patient;
   stimulating, using a third stimulation sequence and via the first stimulation electrode, the one or more areas of the scalp or cranium of the patient, the stimulating configured to generate an MEP;
   recording, via the first recording electrode, a third plurality of resultant waveforms, the third plurality of resultant waveforms representing a facilitated MEP;
   determining, based on the third plurality of resultant waveforms, if a physiological response has occurred; and
   indicating, via a display coupled to the first stimulation electrode and the second stimulation electrode, if the physiological response has occurred.

Embodiment 41: The system of Embodiment 40, wherein the first stimulation sequence comprises a first plurality of stimulation pulses; the second stimulation sequence comprises a single stimulation pulse; and wherein the third stimulation sequence comprises a second plurality of stimulation pulses.

Embodiment 42: The system of Embodiment 40 or 41, wherein the first stimulation sequence is configured to prime the one or more areas of the brain for obtaining the facilitated MEP.

Embodiment 43: The system of any one of Embodiment 40, 41, or 42, wherein the first stimulation electrode is coupled to a scalp of the patient; wherein the second stimulation electrode is coupled to a peripheral or cranial region of interest of the patient, the peripheral or cranial region of interest comprising one or more of a limb, a hand, a wrist, a leg, face, and a foot of the patient.

Embodiment 44: The system of any one of Embodiment 40, 41, 42, or 43, wherein the first stimulation sequence is delivered prior to the third stimulation sequence by an inter-train time interval; and wherein the second stimulation sequence occurs prior, during or after the inter-train time interval.

Embodiment 45: The system of any one of Embodiment 40, 41, 42, 43, or 44, wherein the operations further comprise stimulating using the third stimulation sequence within a period of time after the stimulating using the second stimulation sequence.

Embodiment 46: The system of Embodiment 45, wherein the period of time is a predetermined period of time.

Embodiment 47: The system of Embodiment 45, wherein the period of time is dynamically adjusted based on one or more parameters of the patient and/or a type of surgery being performed on the patient.

Embodiment 48: The system of Embodiment 45, wherein the stimulating using the second stimulation sequence occurs automatically at a predetermined time before, during or between the stimulating using the first stimulation sequence and the stimulating using the third stimulation sequence.

Embodiment 49: The system of any one of Embodiment 40, 41, 42, 43, 44, 45, 46, 47, or 48, wherein the determining comprises comparing the plurality of waveforms to a baseline waveform.

Embodiment 50: The system of any one of Embodiment 40, 41, 42, 43, 44, 45, 46, 47, 48, or 49, wherein the determining if the physiological response has occurred comprises:
comparing one or more features of the MEP to a threshold; and
detecting a presence of the physiological response when the one or more features is greater than or equal to the threshold.

Embodiment 51: The system of any one of Embodiment 40, 41, 42, 43, 44, 45, 46, 47, 48, 49, or 50, wherein the determining the physiological response has occurred comprises:
comparing one or more features of the MEP to a threshold; and
detecting a presence of the physiological response when the one or more features is less than or equal to the threshold.

Embodiment 52: A system for detecting and identifying a patient physiological response, the stimulation system comprising:
at least one processor; and
at least one memory storing instructions which, when executed by the at least one processor, result in operations comprising:
   acquiring one or more first somatosensory evoked potentials (SSEPs), the acquiring comprising stimulating, via a first stimulation electrode, one or more peripheral or cranial nerves of the patient at a region of interest;
   acquiring a motor evoked potential (MEP), the acquiring comprising stimulating, via a second stimulation electrode, one or more areas of the scalp or cranium of the patient at a scalp of the patient;
   determining, based on the MEP, whether a first physiological response has occurred, the first physiological response indicating one or both of whether the patient has motor function in the region of interest and to what extent;
   indicating, via a display coupled to the first stimulation electrode and the second stimulation electrode, that the physiological response has occurred; and
   acquiring one or more second SSEPs, the acquiring comprising stimulating, via the first stimulation electrode, the one or more peripheral nerves of the patient, the one or more second SSEPs indicating continuity of the sensory pathway.

Embodiment 53: A method of facilitating motor evoked potential (MEP) detection, the method comprising:
facilitating a motor evoked potential (MEP) stimulation sequence to obtain an MEP, the facilitating comprising delivering, via a first stimulation electrode, a first stimulation pulse to one or more peripheral or cranial nerves of a patient, the one or more peripheral or cranial nerves located within a region of interest, the first stimulation electrode coupled to the patient in the region of interest;
delivering, after the facilitating and via a second stimulation electrode, the MEP stimulation sequence to one or more areas of the scalp or cranium of the patient to obtain the MEP response, the MEP stimulation sequence comprising a train of stimulation pulses, the second stimulation electrode coupled to a scalp of the patient;
determining, based on the MEP, whether a physiological response has occurred; and
indicating, via a display coupled to the first stimulation electrode and the second stimulation electrode, that the physiological response has occurred;
wherein the facilitating is configured to one or more of: optimize an intensity or number of pulses of the train of stimulation pulses of the MEP stimulation sequence, limit movement of the patient to the region of interest during the delivery of the MEP stimulation, and improve an accuracy of determining whether the physiological response has occurred.

Embodiment 54: A non-transitory computer-readable storage medium including program code, which when executed by at least one data processor, cause operations comprising:
facilitating a motor evoked potential (MEP) stimulation sequence to obtain an MEP, the facilitating comprising delivering, via a first stimulation electrode, a first stimulation pulse to one or more peripheral or cranial nerves of a patient, the one or more peripheral or cranial nerves located within a region of interest, the first stimulation electrode coupled to the patient in the region of interest;
delivering, after the facilitating and via a second stimulation electrode, the MEP stimulation sequence to one or more areas of the scalp or cranium of the patient to obtain the MEP response, the MEP stimulation sequence comprising a train of stimulation pulses, the second stimulation electrode coupled to a scalp of the patient;
determining, based on the MEP, whether a physiological response has occurred; and
indicating, via a display coupled to the first stimulation electrode and the second stimulation electrode, that the physiological response has occurred;
wherein the facilitating is configured to one or more of: optimize an intensity, pulse duration or number of pulses of the train of stimulation pulses of the MEP stimulation sequence, limit movement of the patient to the region of interest during the delivery of the MEP stimulation, and improve an accuracy of determining whether the physiological response has occurred.

Embodiment 55: An apparatus, comprising:
means for facilitating a motor evoked potential (MEP) stimulation sequence to obtain an MEP, the facilitating comprising delivering, via a first stimulation electrode, a first stimulation pulse to one or more peripheral or cranial nerves of a patient, the one or more peripheral or cranial nerves located within a region of interest, the first stimulation electrode coupled to the patient in the region of interest;
means for delivering, after the facilitating and via a second stimulation electrode, the MEP stimulation sequence to one or more areas of the scalp or cranium of the patient to obtain the MEP response, the MEP stimulation sequence comprising a train of stimulation pulses, the second stimulation electrode coupled to a scalp of the patient;
means for determining, based on the MEP, whether a physiological response has occurred; and
means for indicating, via a display coupled to the first stimulation electrode and the second stimulation electrode, that the physiological response has occurred;
wherein the facilitating is configured to one or more of: optimize an intensity pulse duration or number of pulses of the train of stimulation pulses of the MEP stimulation sequence, limit movement of the patient to the region of interest during the delivery of the MEP stimulation, and improve an accuracy of determining whether the physiological response has occurred.

Embodiment 56: The apparatus of Embodiment 55, comprising: means for performing any of the functions recited in any one of Embodiment 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, or 52.

Embodiment 57: A method of performing a spinal procedure, the method comprising:
facilitating a motor evoked potential (MEP) stimulation sequence to obtain an MEP, the facilitating comprising delivering, via a first stimulation electrode, a first stimulation pulse to one or more peripheral nerves of a patient, the one or more peripheral nerves located within a region of interest, the first stimulation electrode coupled to the patient in the region of interest;
delivering, after the facilitating and via a second stimulation electrode, the MEP stimulation sequence to one or more areas of the scalp or cranium of the patient to obtain the MEP response, the MEP stimulation sequence comprising a train of stimulation pulses, the second stimulation electrode coupled to a scalp of the patient;
determining, based on the MEP, whether a physiological response has occurred; and
indicating, via a display coupled to the first stimulation electrode and the second stimulation electrode, that the physiological response has occurred;
wherein the facilitating is configured to one or more of: optimize an intensity or number of pulses of the train of stimulation pulses of the MEP stimulation sequence, limit movement of the patient to the region of interest during the delivery of the MEP stimulation, and improve an accuracy of determining whether the physiological response has occurred.

Embodiment 58: A method of reducing movement of a patient during spinal surgery, the method comprising:
facilitating a motor evoked potential (MEP) stimulation sequence to obtain an MEP, the facilitating comprising delivering, via a first stimulation electrode, a first stimulation pulse to one or more peripheral or cranial nerves of a patient, the one or more peripheral or cranial nerves located within a region of interest, the first stimulation electrode coupled to the patient in the region of interest;
delivering, after the facilitating and via a second stimulation electrode, the MEP stimulation sequence to one or more areas of the scalp or cranium of the patient to obtain the MEP response, the MEP stimulation sequence comprising a train of stimulation pulses, the second stimulation electrode coupled to a scalp of the patient;
determining, based on the MEP, whether a physiological response has occurred; and
indicating, via a display coupled to the first stimulation electrode and the second stimulation electrode, that the physiological response has occurred;
wherein the facilitating is configured to one or more of: optimize an intensity or number of pulses of the train of stimulation pulses of the MEP stimulation sequence, limit movement of the patient to the region of interest during the delivery of the MEP stimulation, and improve an accuracy of determining whether the physiological response has occurred.

Embodiment 59: A method of automatically facilitating the acquisition of a motor evoked potential (MEP) of a patient undergoing a surgical procedure, the method comprising:
providing a facilitating stimulation via a first peripherally positioned electrode;
providing a stimulation via a second cranially positioned electrode;
recording an MEP resulting from the stimulation via a second recording electrode;
automatically determining—via a processor—whether the MEP is acceptable;
using the processor to analyze the MEP to determine a nerve health of the patient; and
displaying—via a display—at least one of the nerve health and the MEP.

Embodiment 60: The method of Embodiment 59, further comprising recording a somatosensory evoked potential (SEP) resulting from the facilitating stimulation.

Embodiment 61: The method of Embodiment 60, further comprising automatically determining—via the processor—whether the SEP is acceptable.

Embodiment 62: The method of Embodiment 61, further comprising using the processor to analyze the SEP to determine the nerve health of the patient.

Embodiment 63: The method of Embodiment 62, further comprising displaying—via the display—at least one of the nerve health and the SEP.

Embodiment 64: The method of any one of Embodiment 59, 60, 61, 62, or 63, further comprising—prior to providing the facilitating stimulation—providing a conditioning stimulation via the second cranially positioned electrode.

Embodiment 65: The method of Embodiment 64, further comprising recording a conditioned MEP resulting from the conditioning stimulation.

Embodiment 66: The method of Embodiment 65, further comprising automatically determining—via the processor—whether the conditioned MEP is acceptable.

Embodiment 67: The method of Embodiment 66, further comprising using the processor to analyze the conditioned MEP to determine the nerve health of the patient.

Embodiment 68: The method of Embodiment 67, further comprising displaying—via the display—at least one of the nerve health and the conditioned MEP.

Embodiment 69: The method of any one of Embodiment 59, 60, 61, 62, 63, 64, 65, 66, 67, or 68, further comprising establishing automatically—via the processor—a baseline MEP that is either the MEP or the conditioned MEP.

Embodiment 70: The method of Embodiment 69, further comprising:
providing a subsequent stimulation via the second cranially positioned electrode;
recording a subsequent MEP resulting from the subsequent stimulation; and
using the processor to analyze the subsequent MEP to determine the nerve health of the patient by comparing an aspect of the subsequent MEP to an aspect of the baseline MEP.

Embodiment 71: The method of Embodiment 70, wherein the aspect includes at least one of amplitude, morphology, latency, and signal to noise ratio.

Embodiment 72: The method of Embodiment 70 or 71, wherein—based on the nerve health of the patient as determined by comparing the subsequent MEP to the baseline MEP—the processor generates an alert indicating a problem in the nerve health, modifies the baseline MEP based on the subsequent MEP, or generates an updated indication of nerve health to be displayed by the display.

Embodiment 73: The method of any one of Embodiment 59, 60, 61, 62, 63, 64, 65, 66, 67, 68, 69, 70, 71, or 72, wherein the facilitating stimulation is provided via the first peripherally positioned electrode to a region of interest of the patient.

Embodiment 74: The method of Embodiment 73, wherein the region of interest one or more of a limb, a hand, a wrist, or a leg of the patient.

Embodiment 75: The method of Embodiment 73, or 74, wherein the facilitating stimulation is provided via a second peripherally positioned electrode that is located in a region of interest that is distinct from the region of interest where the first peripherally positioned electrode is located.

Embodiment 76: The method of any one of Embodiments 59, 60, 61, 62, 63, 64, 65, 66, 67, 68, 69, 70, 71, 72, 73, 74, or 75, wherein-because of the facilitating stimulation—the MEP is preferentially directed to a region of interest.

Embodiment 77: The method of any one of Embodiments 59, 60, 61, 62, 63, 64, 65, 66, 67, 68, 69, 70, 71, 72, 73, 74, 75, or 76, wherein-because of the facilitating stimulation—the MEP is more reliable than without the facilitating stimulation.

Embodiment 78: The method of any one of Embodiments 59, 60, 61, 62, 63, 64, 65, 66, 67, 68, 69, 70, 71, 72, 73, 74, 75, 76, or 77, wherein-because of the facilitating stimulation—the stimulation via the second cranially positioned electrode has a lower intensity than would otherwise be required to obtain a suitable MEP.

Although the disclosure, including the figures, described herein may describe and/or exemplify different variations separately, it should be understood that all or some, or components of them, may be combined.

Although various illustrative implementations are described above, any of a number of changes may be made to various implementations. For example, the order in which various described method steps are performed may often be changed in alternative implementations, and in other alternative implementations one or more method steps may be skipped altogether. Optional features of various device and system implementations may be included in some implementations and not in others. Therefore, the foregoing description is provided primarily for exemplary purposes and should not be interpreted to limit the scope of the claims.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other implementations. References to a structure or feature that is disposed “adjacent” another feature may have portions that overlap or underlie the adjacent feature.

Terminology used herein is for the purpose of describing particular implementations only and is not intended to be limiting. For example, as used herein, the singular forms “a,” “an,” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as, for example, “under,” “below,” “lower,” “over,” “upper,” and the like, may be used herein for ease of description to describe one element or one feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly,” “downwardly,” “vertical,” “horizontal,” and the like are used herein for the purpose of explanation only unless specifically indicated otherwise.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings provided herein.

Throughout this specification and the claims which follow, unless the context requires otherwise, the word “comprise” and variations such as “comprises” and “comprising” means various components can be co-jointly employed in the methods and articles (e.g., compositions and apparatuses including device and methods). For example, the term “comprising” will be understood to imply the inclusion of any stated elements or steps but not the exclusion of any other elements or steps.

As used herein in the specification and claims, including as used in the examples and unless otherwise expressly specified, all numbers may be read as if prefaced by the word “about” or “approximately,” even if the term does not expressly appear. The phrase “about” “or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical values given herein should also be understood to include about or approximately that value, unless the context indicates otherwise.

The examples and illustrations included herein show, by way of illustration and not of limitation, specific implementations in which the subject matter may be practiced. As mentioned, other implementations may be utilized and derived there from, such that structural and logical substitutions and changes may be made without departing from the scope of this disclosure. Although specific implementations have been illustrated and described herein, any arrangement calculated to achieve the same purpose may be substituted for the specific implementations shown. This disclosure is intended to cover any and all adaptations or variations of various implementations. Combinations of the above implementations, and other implementations not specifically described herein, are possible.

In the descriptions above and in the claims, phrases such as, for example, “at least one of” or “one or more of” may occur followed by a conjunctive list of elements or features. The term “and/or” may also occur in a list of two or more elements or features. Unless otherwise implicitly or explicitly contradicted by the context in which it is used, such a phrase is intended to mean any of the listed elements or features individually or any of the recited elements or features in combination with any of the other recited elements or features. For example, the phrases “at least one of A and B;” “one or more of A and B;” and “A and/or B” are each intended to mean “A alone, B alone, or A and B together.” A similar interpretation is also intended for lists including three or more items. For example, the phrases “at least one of A, B, and C;” “one or more of A, B, and C;” and “A, B, and/or C” are each intended to mean “A alone, B alone, C alone, A and B together, A and C together, B and C together, or A and B and C together.” Use of the term “based on,” above and in the claims is intended to mean, “based at least in part on,” such that an unrecited feature or element is also permissible.

As used herein a “user interface” (also referred to as an interactive user interface, a graphical user interface or a UI) may refer to a network based interface including data fields and/or other control elements for receiving input signals or providing electronic information and/or for providing information to the user in response to any received input signals. Control elements may include dials, buttons, icons, selectable areas, or other perceivable indicia presented via the UI that, when interacted with (e.g., clicked, touched, selected, etc.), initiates an exchange of data for the device presenting the UI. A UI may be implemented in whole or in part using technologies such as hyper-text mark-up language (HTML), FLASH™, JAVA™, .NET™, C, C++, web services, or rich site summary (RSS). In some implementations, a UI may be included in a stand-alone client (for example, thick client, fat client) configured to communicate (e.g., send or receive data) in accordance with one or more of the aspects described. The communication may be to or from a medical device or server in communication therewith.

As used herein, the terms “determine” or “determining” encompass a wide variety of actions. For example, “determining” may include calculating, computing, processing, deriving, generating, obtaining, looking up (e.g., looking up in a table, a database or another data structure), ascertaining and the like via a hardware element without user intervention. Also, “determining” may include receiving (e.g., receiving information), accessing (e.g., accessing data in a memory) and the like via a hardware element without user intervention. “Determining” may include resolving, selecting, choosing, establishing, and the like via a hardware element without user intervention.

As used herein, the terms “provide” or “providing” encompass a wide variety of actions. For example, “providing” may include storing a value in a location of a storage device for subsequent retrieval, transmitting a value directly to the recipient via at least one wired or wireless communication medium, transmitting or storing a reference to a value, and the like. “Providing” may also include encoding, decoding, encrypting, decrypting, validating, verifying, and the like via a hardware element.

As used herein, the term “message” encompasses a wide variety of formats for communicating (e.g., transmitting or receiving) information. A message may include a machine readable aggregation of information such as an XML document, fixed field message, comma separated message, JSON, a custom protocol, or the like. A message may, in some implementations, include a signal utilized to transmit one or more representations of the information. While recited in the singular, it will be understood that a message may be composed, transmitted, stored, received, etc. in multiple parts.

As used herein, the term “selectively” or “selective” may encompass a wide variety of actions. For example, a “selective” process may include determining one option from multiple options. A “selective” process may include one or more of: dynamically determined inputs, preconfigured inputs, or user-initiated inputs for making the determination. In some implementations, an n-input switch may be included to provide selective functionality where n is the number of inputs used to make the selection.

As user herein, the terms “correspond” or “corresponding” encompasses a structural, functional, quantitative and/or qualitative correlation or relationship between two or more objects, data sets, information and/or the like, preferably where the correspondence or relationship may be used to translate one or more of the two or more objects, data sets, information and/or the like so to appear to be the same or equal. Correspondence may be assessed using one or more of a threshold, a value range, fuzzy logic, pattern matching, a machine learning assessment model, or combinations thereof.

In any embodiment, data generated or detected can be forwarded to a “remote” device or location, where “remote,” means a location or device other than the location or device at which the program is executed. For example, a remote location could be another location (e.g., office, lab, etc.) in the same city, another location in a different city, another location in a different state, another location in a different country, etc. As such, when one item is indicated as being “remote” from another, what is meant is that the two items can be in the same room but separated, or at least in different rooms or different buildings, and can be at least one mile, ten miles, or at least one hundred miles apart. “Communicating” information references transmitting the data representing that information as electrical signals over a suitable communication channel (e.g., a private or public network). “Forwarding” an item refers to any means of getting that item from one location to the next, whether by physically transporting that item or otherwise (where that is possible) and includes, at least in the case of data, physically transporting a medium carrying the data or communicating the data. Examples of communicating media include radio or infra-red transmission channels as well as a network connection to another computer or networked device, and the internet or including email transmissions and information recorded on websites and the like.

The examples and illustrations included herein show, by way of illustration and not of limitation, specific implementations in which the subject matter may be practiced. As mentioned, other implementations may be utilized and derived there from, such that structural and logical substitutions and changes may be made without departing from the scope of this disclosure. Such implementations of the inventive subject matter may be referred to herein individually or collectively by the term “invention” merely for convenience and without intending to voluntarily limit the scope of this application to any single invention or inventive concept, if more than one is, in fact, disclosed. Thus, although specific implementations have been illustrated and described herein, any arrangement calculated to achieve the same purpose may be substituted for the specific implementations shown. This disclosure is intended to cover any and all adaptations or variations of various implementations. Combinations of the above implementations, and other implementations not specifically described herein, will be apparent to those of skill in the art upon reviewing the above description.

What is claimed is:

1. A stimulation system for detecting and identifying a patient physiological response, the stimulation system comprising:
at least one processor; and
at least one memory storing instructions which, when executed by the at least one processor, result in operations comprising:
facilitating a motor evoked potential (MEP) stimulation sequence to obtain an MEP, the facilitating comprising delivering, via a first stimulation electrode, a first stimulation pulse to one or more peripheral or cranial nerves of a patient, the one or more peripheral or cranial nerves located within a region of interest, the first stimulation electrode coupled to the patient in the region of interest;
delivering, after the facilitating and via a second stimulation electrode, the MEP stimulation sequence to one or more areas of the of the patient to obtain the MEP response, the MEP stimulation sequence comprising a train of stimulation pulses, the second stimulation electrode coupled to a scalp of the patient;
determining, based on the MEP, whether a physiological response has occurred; and
indicating, via a display coupled to the first stimulation electrode and the second stimulation electrode, that the physiological response has occurred;
wherein the facilitating is configured to one or more of: optimizing an intensity or pulse duration or number of pulses of the train of stimulation pulses of the MEP stimulation sequence, limit movement of the patient to the region of interest during the delivery of the MEP stimulation, and improve an accuracy of determining whether the physiological response has occurred.

2. The system of claim 1, wherein the first stimulation pulse comprises a single stimulation pulse.

3. The system of claim 1, wherein delivering the first stimulation pulse to the one or more peripheral nerves of the patient is configured be delivered by an independent stimulator or one used for SSEP stimulation.

4. The system of claim 1, wherein the operations further comprise delivering, prior, during or after to the facilitating and via the second stimulation electrode, a second MEP stimulation sequence to the one or more areas of the scalp or cranium of the patient, the second MEP stimulation sequence comprising a second train of stimulation pulses.

5. The system of claim 4, wherein the second MEP stimulation sequence is delivered prior to the MEP stimulation sequence by an inter-train time interval; and wherein the facilitating occurs prior, during, or after the inter-train time interval.

6. The system claim 1, wherein the operations further comprise delivering the MEP sequence within a period of time after the facilitation of the MEP stimulation sequence.

7. The system of claim 6, wherein the period of time is a predetermined period of time.

8. The system of claim 6, wherein the period of time is dynamically adjusted based on one or more parameters of the patient and/or a type of surgery being performed on the patient.

9. The system of claim 6, wherein the facilitating occurs automatically within the period of time prior to the delivering the MEP stimulation sequence.

10. The system of claim 1, wherein the facilitating is manually initiated prior to the delivering the MEP stimulation sequence.

11. The system of claim 1, wherein the facilitating is automatically initiated by the system in response to one or more inputs from an SSEP monitoring system, an EMG monitoring system, a heart rate monitoring system, a blood pressure monitoring system, or other patient monitoring system.

12. The system of claim 11, wherein one or more of the SSEP monitoring system, the EMG monitoring system, the heart rate monitoring system, the blood pressure monitoring system, and the other patient monitoring system is contained within or is a part of the stimulation system herein claimed.

13. The system of claim 1, wherein the MEP comprises one or more waveforms.

14. The system of claim 1, wherein the processor stores the MEP as a baseline waveform.

15. The system of claim 14, wherein the determining comprises comparing the one or more waveforms of the MEP to the baseline waveform.

16. The system of claim 1, wherein the determining whether the physiological response has occurred comprises:
comparing one or more features of the MEP to a threshold; and/or
detecting a presence of the physiological response when the one or more features is greater than or equal to the threshold.

17. The system of claim 1, wherein the determining whether the physiological response has occurred comprises:
comparing one or more features of the MEP to a threshold; and/or
detecting a presence of the physiological response when the one or more features is less than or equal to the threshold.

18. The system claim 1, further comprising:
a somatosensory evoked potential (SSEP) stimulation/acquisition system configured to use the same facilitation electrode or electrodes as the MEP stimulation sequence, the SSEP stimulation/acquisition system configured to acquire one or more SSEPs during or prior to delivery of the first stimulation pulse; and
an MEP acquisition system configured to deliver the MEP stimulation sequence.

19. The system of claim 1, further comprising:
the stimulation electrode; and
the recording electrode.

20. The system of claim 1, wherein the second stimulation electrode comprises four or more electrodes.
